(19) United States
(12) Patent Application Publication
(10) Pub. No.: US 2020/0000525 A1
(43) Pub. Date: Jan. 2, 2020

(54) INTERNAL ULTRASOUND ASSISTED LOCAL THERAPEUTIC DELIVERY

(71) Applicant: KONINKLIJKE PHILIPS N.V., EINDHOVEN (NL)

(72) Inventors: Jeremy STIGALL, CARLSBAD, CA (US); John Arthur PEDERSEN, EDEN PRAIRIE, MN (US); Mary GAO, SAN DIEGO, CA (US)

(21) Appl. No.: 16/454,484

(22) Filed: Jun. 27, 2019

Related U.S. Application Data
(60) Provisional application No. 62/691,535, filed on Jun. 28, 2018.

(51) Int. Cl. A61B 34/20

(52) U.S. Cl. CPC A61B 34/20; A61B 2090/3735; A61B 2034/2063

(57) ABSTRACT

An imaging system and device used either alone or in conjunction with an intravascular treatment device to deliver a therapeutic agent to soft tissue within a subject.

### CROSS-REFERENCE TO RELATED APPLICATION

This application claims the benefit of U.S. Provisional Application No. 62/691,535 filed Jun. 28, 2018. This application is hereby incorporated by reference herein.

### FIELD OF THE DISCLOSURE

The present disclosure relates generally to devices, methods and systems associated with ultrasound and local delivery of therapeutic agents to treat soft tissue via intravascular devices.

### BACKGROUND

Intravascular catheters may be used to successfully treat a variety of medical problems including chronic total occlusions, thrombosis, hypertension, and atherosclerosis. These catheters have the potential to save lives when used effectively and efficiently.

Intravascular treatment via catheter, especially in tortuous vasculature, requires a high degree of precision. Current devices can prove inaccurate, leading to ineffective treatment by missing the treatment target or causing complications such as trauma to, or perforation of, the vessel wall. Accordingly, safe treatment with catheters can require the use, and exchange, of multiple separate devices for tasks such as intravascular imaging, steering, and treatment. Each of these individual devices must be advanced through the vasculature, removed, and replaced with the next device. Some procedures may require multiple exchanges such as insertion and removal of an imaging device for pre-treatment navigation and post-treatment verification. Patient risk is increased as each device exchange introduces additional opportunities for vascular trauma and other complications. Furthermore, many external imaging techniques require exposure to x-rays and other potentially harmful radiation and prolonging procedures likewise prolongs the exposure.

### SUMMARY

Accordingly, there is a need for a device, method and/or system for detecting, monitoring and/or treatment that determines the size and other characteristics of soft tissue so as

to accurately, effectively and proportionately deliver a corresponding amount of a therapeutic agent to the soft tissue based on the size and other characteristics of the soft tissue. The present disclosure discusses such a detection, monitoring and/or treatment device, method and/or system. An example of an intravascular treatment system in accordance with this disclosure includes a catheter comprising a distal portion and a proximal portion, an imaging apparatus disposed at the distal portion of the catheter and configured to image a location within a subject's soft tissue disposed externally of vasculature, the imaging apparatus producing an image signal, and a first lumen comprising a first exit port disposed at the distal portion of the catheter, wherein the first lumen is configures to receive a guidewire, and a needle slidably disposed within the catheter and substantially parallel to at least a portion of the first lumen of the catheter, wherein the needle comprises a second lumen and a second exit port, and a controller for receiving the image signal, the controller comprising a non-transitory computer-readable medium containing instructions that, when executed, cause one or more processors to image the subject's soft tissue disposed externally of vasculature using the image signal identify a target area within the subject's soft tissue, translate the needle relative to the catheter and inserting the needle through the vasculature to the target area, and deliver a therapeutic agent to the target area through the needle.

The system according to the previous paragraph, wherein the non-transitory computer-readable medium's instructions for identifying the target area comprise instructions that, when executed, cause one or more processors to determine a type of tissue within the target area.

The system according to any of the previous paragraphs, wherein the non-transitory computer-readable medium's instructions for identifying the target area comprise instructions that, when executed, cause one or more processors to determine a location or position of the target area within the subject's soft tissue.

The system according to any of the previous paragraphs, wherein the location or position of the target area comprises distance.

The system according to any of the previous paragraphs, wherein the distance is relative to another portion of the subject's soft tissue.

The system according to any of the previous paragraphs, wherein the imaging apparatus produces a plurality of image signals.

The system according to any of the previous paragraphs, wherein the distance is determined by determining the time of flight of an echo signal, wherein the echo signal is a derivative signal of one of the image signals.

The system according to any of the previous paragraphs, wherein the distance is determined by determining the time of flight of another echo signal, wherein the other echo signal is a derivative signal of a second of the image signal.

The system according to any of the previous paragraphs, wherein the non-transitory computer-readable medium's instructions for identifying the target area comprise instructions that, when executed, cause one or more processors to determine the thickness of the soft tissue.

The system according to any of the previous paragraphs, wherein the non-transitory computer-readable medium's instructions for identifying the target area comprise instructions that, when executed, cause one or more processors to determine the thickness of the soft tissue using the difference in time of flight between the first echo signal and the second echo signal.

The system according to any of the previous paragraphs, wherein the non-transitory computer-readable medium's instructions for identifying the target area comprise instructions that, when executed, cause one or more processors to identify a size of the target area.

The system according to any of the previous paragraphs, wherein the non-transitory computer-readable medium's instructions for identifying the target area comprise instructions that, when executed, cause one or more processors to identify a density of the target area.

The system according to any of the previous paragraphs, wherein the instructions for delivering the therapeutic agent to the target area through the needle comprise instructions that, when executed, cause one or more processors to deliver an amount of therapeutic agent based upon at least one of a size of the target area and a density of the target area.

The system according to any of the previous paragraphs, wherein the image signal is produced from a transducer.

The system according to any of the previous paragraphs, wherein the transducer produces energy between 500 kilohertz (KHz) to 30 megahertz (MHz).

This disclosure generally relates to medical devices, systems, and methods for providing intravascular treatment with dual guidewire lumens with imaging capabilities near the distal exit ports of at least one lumen. By providing intravascular imaging capability directly at a lumen exit port, the need to exchange imaging and treatment catheters is avoided. Additionally, the two lumens allow easy guidewire exchange as one guidewire and lumen may be used for support while another guidewire is advanced in the other lumen. The two lumens also improve steering capability by allowing the use of shaped wires for side branch access and navigating bifurcations. Additionally, substantially parallel orientation of the two lumens at the distal portion of the catheter provides improved centering of the catheter body during use. The elements of the inventive catheters can provide real-time imaging of the treatment site during treatment and may reduce the need for exchanges of separate devices, minimizing the associated risk of vascular trauma and reducing procedure time.

By setting the distal exits of both lumens near the location of an imaging apparatus, the catheter allows a user to make navigation and therapy delivery decisions with reference to images obtained from the delivery location. Locating one or more of the exit ports of a dual lumen catheter near an imaging apparatus can allow accurate delivery of an appropriate treatment to a target area with minimal adjustments. A distally located imaging apparatus may also be used to monitor and verify the effectiveness of treatment during and after delivery.

Guidewire lumens may be of any suitable type such as over-the-wire (OTW), which allow for easy exchange of guidewires, or rapid exchange (RX), which can be more quickly threaded and require shorter guidewires. In certain embodiments, catheters of the present disclosure may include one OTW guidewire lumen and one RX guidewire lumen, taking advantage of both types. Guidewire lumens may have exit ports on the distal portion of the catheter and within a short distance of the imaging apparatus. In various embodiments, one or both of the lumens may pass through the imaging apparatus. Lumen exit ports may be proximal or distal to the imaging apparatus and may be adjacent one another or offset. Exit ports may be flat or may be skive cut to form an angle with the distal portion of the catheter body, allowing for easier passage of the catheter through the vasculature.

An imaging apparatus may include an ultrasound transducer as part of an intravascular ultrasound (IVUS) assembly. In some embodiments, the imaging apparatus may be an optical coherence tomography (OCT) imaging apparatus. Optionally, the distal portion of the catheter may include a functional measurement sensor configured to sense parameters such as pressure, velocity, or Doppler velocity. The distal portion may include a transducer support configured to support a variety of imaging assemblies. In certain embodiments, imaging assemblies may be interchangeably affixed to the transducer support.

To aid in visualization and orientation of the distal portion of the body and the exit ports within vasculature, the distal portion of the catheter body may include a pattern of radiopaque or other markers that may be externally monitored by, for example, x-ray. Catheter bodies may include a variety of features to efficiently transfer axial torque applied at the proximal end of the catheter to the distal end of the catheter, easing manipulation of the distal end during navigation or treatment delivery. Catheters of the present disclosure may be compatible with automated body lumen measurement software such as VH® IVUS from Volcano Corporation (San Diego, Calif.), image highlighting software for blood, plague, and foreign body differentiation such as ChromaFlo® from Volcano Corporation (San Diego, Calif.), and software for correlating a single view from IVUS and angiogram images such as SyncVision™ from Volcano Corporation (San Diego, Calif.).

Catheters of the present disclosure may be used for crossing a chronic total occlusion, tissue ablation, thrombolysis, drug dispersion, aspiration, echogenic injection, to navigate through bifurcations or for side branch access. The combination of two guidewire lumens, external orientation tracking, efficient axial torque transfer, and localized intravascular imaging at the lumen exit ports provides delivery of catheter based treatments that are quicker, safer, and more accurate and effective than provided by current catheters. Catheters of the present disclosure may include centering mechanisms including inflatable balloons, or collapsible members (e.g., a sheathable nitinol basket) of various shapes and sizes disposed near the distal end of the catheter and the first and/or second exit ports. Centering mechanisms can be configured to interact with a lumen wall in order to center the first and/or second exit ports within a cross-section of the vessel, artery, or other lumen. Catheters of the present disclosure may include perfusion holes.

In certain aspects, the present disclosure relates to an intravascular treatment catheter having an elongated body with a distal portion and a proximal portion. The catheter has an imaging apparatus at the distal portion of the body configured to image a location within vasculature. The catheter body includes a first guidewire lumen with a first exit port disposed at the distal portion of the body and a second guidewire lumen substantially parallel to the first at least at the distal portion of the body with a second exit port also disposed at the distal portion of the body.

The imaging apparatus can include an ultrasound transducer which may be an intravascular ultrasound (IVUS) imaging apparatus with a micromachined ultrasonic transducer. In some embodiments, the imaging apparatus may include an optical coherence tomography (OCT) imaging apparatus. The catheter may also include a functional measurement sensor such as a pressure sensor, a velocity sensor, a Doppler velocity sensor, or an optical sensor, at the distal portion of the body.

In various embodiments, the first guidewire lumen of the catheter may be an over-the-wire guidewire lumen and the second guidewire lumen may be a rapid exchange guidewire lumen. The first exit port and the second exit port may be offset from each other and either or both may be located within 5 cm of the imaging apparatus. In certain embodiments, at least one of the first and second exit ports forms an obtuse angle with a line tangential to the distal portion of the elongated body.

The imaging apparatus may be located distal to the first exit port. In some configurations, the catheter may include a shaft, a braided material, or a coiled material or may be otherwise configured to transmit axial torque applied at the proximal portion of the body to the distal portion of the body.

In certain embodiments, the imaging apparatus is disposed around the second guidewire lumen. The distal portion of the catheter body may include a pattern configured to show an orientation of the distal portion of the body under x-ray imaging. The catheter may include a third lumen and a microcable therein with the microcable extending from the imaging apparatus to the proximal portion of the catheter and in electronic communication with the imaging apparatus.

In certain aspects, the present disclosure provides methods of delivering intravascular treatment. The methods include advancing a first guidewire substantially to a portion of a vessel to be treated, advancing an intravascular treatment catheter over the first guidewire, imaging the portion of the vessel to be treated, and delivering treatment. The intravascular treatment catheter includes an elongated body having a distal portion and a proximal portion, and an imaging apparatus at the distal portion. The imaging apparatus is configured to image the portion of the vessel to be treated. The intravascular treatment catheter includes a first guidewire lumen having a first exit port and a second guidewire lumen substantially parallel to the first guidewire lumen at least at the distal portion of the body and having a second exit port. Both the first and second exit ports may be at the distal portion of the catheter body.

Methods of the present disclosure may include steering the intravascular treatment catheter through one selected branch of a bifurcation in the vasculature. Steering the catheter may be accomplished by advancing the first guidewire to the bifurcation, advancing the catheter over the first guidewire to the bifurcation, imaging the bifurcation and then selecting a shaped guidewire configured to enter the desired branch of the bifurcation. The shaped guidewire is advanced through the second guidewire lumen and into the desired branch of the bifurcation, after which the catheter may be advanced over the shaped guidewire into the desired branch.

In some embodiments, methods can include treating a chronic total occlusion by advancing the catheter along the first guidewire to the chronic total occlusion. Preferably, the catheter includes a functional measurement sensor configured to sense pressure and located at the distal portion of the body. The functional measurement sensor may be used to verify position of the distal portion of the body at the chronic total occlusion by sensing a change in pressure. Methods of the present disclosure may include using the first guidewire for support while crossing the chronic total occlusion with a second guidewire advanced through the second guidewire lumen; and delivering a therapy to the chronic total occlusion.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “logic” or ““control logic” as used herein may include software and/or firmware executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion on and/or in conjunction with computer-readable medium and would remain in accordance with the embodiments herein disclosed.

The term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 shows a dual lumen catheter assembly.

FIG. 2 shows a distal portion of a dual lumen catheter assembly.

FIG. 3 shows a dual lumen tracking tip with imaging apparatus support.

FIG. 4 shows a dual lumen tracking tip with an extended imaging apparatus support and a cut-out at the imaging plane of the imaging apparatus.

FIG. 5 shows a dual lumen imaging apparatus support with a functional measurement sensor.

FIG. 6 shows a dual lumen imaging apparatus support with offset exit ports and a flattened surface after the proximal of the two exit ports.

FIG. 7 shows a dual lumen imaging apparatus support with offset exit ports, an angle cut exit port and a glue port hole.

FIG. 8 shows a dual lumen imaging apparatus support with offset exit ports and rounded exit port and a functional measurement sensor.

FIG. 9 shows a dual lumen imaging apparatus support with guidewires therethrough and a single lumen short tip.

FIG. 10 shows a dual lumen imaging apparatus support with guidewires therethrough and a dual lumen extended tip.

FIGS. 11A-11D show dual lumen imaging apparatuses with a first exit port having various configurations relative to an imaging apparatus.

FIG. 12 shows a dual lumen imaging apparatus with perfusion holes and a multiple balloon centering mechanism.

FIG. 13 shows a head-on view of the distal end of a dual lumen imaging apparatus with a multiple balloon centering mechanism.

FIG. 14 shows a dual lumen imaging apparatus support with perfusion holes.

FIG. 15 shows a dual lumen imaging apparatus with perfusion holes and a spiral-shaped balloon centering mechanism.

FIG. 16 shows a dual lumen imaging apparatus with perfusion holes and a balloon centering mechanism having a cross-sectional shape of a segmented circle with multiple open sections.

FIG. 17 shows a head-on view of the distal end of a dual lumen imaging apparatus with a balloon centering mechanism having a cross-sectional shape of a segmented circle with multiple open sections.

FIG. 18 shows a data-acquisition system, a patient monitoring system, and/or a therapeutic and control system including a controller, an intravascular-ultrasound (IVUS) catheter having a transducer, an external ultrasound device having a transducer, and an intravascular therapeutic needle that can be used alone or in conjunction with the IVUS catheter.

FIG. 19 shows a block diagram depicting an illustrative computing device, in accordance with various embodiments of the present disclosure.

FIG. 20 shows a block diagram depicting an illustrative a data-acquisition system, a patient monitoring system, and/or a therapeutic and control system, in accordance with embodiments of the present disclosure.

FIG. 21 shows a data-acquisition system, a patient monitoring system, and/or a therapeutic and control system including a controller, an intravascular-ultrasound (IVUS) catheter having a transducer inserted within a patient's vasculature, an external ultrasound device having a transducer, and an intravascular therapeutic needle inserted within a patient's vasculature.

FIG. 22 shows an intravascular-ultrasound (IVUS) catheter having a transducer inside a patient's vasculature.

FIG. 23 shows an intravascular-ultrasound (IVUS) catheter having a transducer inside a patient's vasculature and receiving ultrasound data as backscattered from vascular tissue.

FIG. 24 shows an intravascular-ultrasound (IVUS) catheter having a transducer inside a patient's vasculature and receiving ultrasound data as backscattered from soft tissue within a patient exterior of the vasculature and exterior of the vascular tissue.

FIG. 25 shows an external imaging apparatus having a transducer located exterior of the patient and receiving ultrasound data as backscattered from soft tissue within a patient exterior of the vasculature and exterior of the vascular tissue.

FIG. 26 shows a dual lumen imaging apparatuses with a first exit port having a needle extending therefrom and through the vasculature and into soft tissue of a patient exterior of the vasculature and exterior of the vascular tissue, wherein the needle delivers a therapeutic agent into a target within the soft tissue.

FIG. 27 shows an external imaging apparatus and a dual lumen imaging apparatuses with a first exit port having a needle extending therefrom and through the vasculature and into soft tissue of a patient exterior of the vasculature and exterior of the vascular tissue, wherein the needle delivers a therapeutic agent into a target within the soft issue.

FIG. 28 is a block diagram or flow chart of operating and/or using the device(s) discussed herein, such as the device(s) illustrated in FIG. 26.

FIG. 29 is a block diagram or flow chart of operating and/or using the device(s) discussed herein, such as the device(s) illustrated in FIG. 27.

FIG. 30 is a block diagram or flow chart of operating and/or using the device(s) discussed herein, such as the device(s) illustrated in FIG. 26 and FIG. 27.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The present disclosure generally relates to dual lumen intravascular treatment catheters with an imaging apparatus near the distal exits of both lumens, allowing a user to make steering and treatment decisions based on direct imaging from the lumen exits and accurately deliver the appropriate treatment to the target area with very minimal adjustments. The presence of two lumens allows for increased support, improved catheter centering, and improved steering through the use and easy exchange of various shaped guidewires. Additional features of the catheter may include various means of increased torsional rigidity for better axial torque transmission to the distal end of the catheter. Catheters of the present disclosure may also include a pattern of radiopaque markers or other means for external determination of catheter orientation at the distal portion. In certain instances the distal portion of the catheter may include additional functional measurement sensors to aid in navigation and the accurate delivery of treatment.

Catheter Body

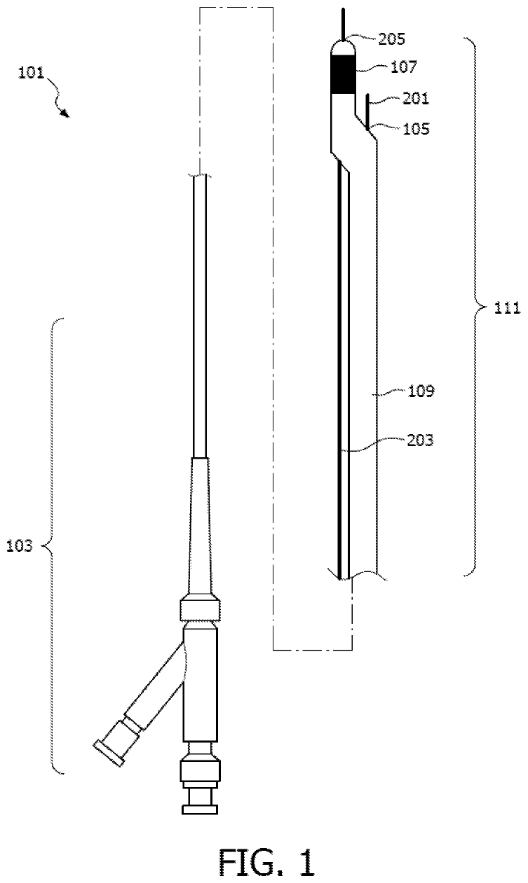

FIG. 1 shows a catheter 101 with an elongated body 109 having an OTW guidewire 201 and a RX guidewire 203 disposed within the elongated body 109 in first and second guidewire lumen (not shown) respectively and exiting through a first exit port 105 and a second exit port 205 respectively. Catheter 101 generally includes a proximal portion 103 extending to a distal portion 111. An imaging apparatus 107, such as an ultrasound transducer, may be located at the distal portion 111.

The intravascular catheter is configured for intraluminal introduction to a target body lumen. The dimensions and other physical characteristics of the catheter bodies will vary significantly depending on the body lumen that is to be accessed. Catheters of the present disclosure may include two or more lumens. Lumens may be a variety of types including “over-the-wire” (OTW) where a guidewire channel extends fully through the catheter body or for “rapid exchange” (RX) where the guidewire channel extends only through a distal portion of the catheter body. In an exemplary embodiment, as shown in FIG. 1, a catheter of the present disclosure may include at least one RX and at least one OTW lumen to exploit the unique advantages of each type of guidewire lumen.

The catheter may include additional lumens to house microcables in electronic communication with the imaging apparatus, support or torsion members, drive shafts or cables, or other purposes. The catheter may also include an additional lumen and luer and/or adaptor for introduction of a therapeutic agent into the catheter for delivery by the needle to a targeted tissue site.

The dual lumens may be substantially parallel over the course of the catheter body. In certain embodiments, the lumens may be substantially parallel only at the distal portion of the catheter and/or at their respective exit ports.

Catheter bodies intended for intravascular introduction will typically have a length in the range from 50 cm to 200 cm and an outer diameter in the range from 1 French to 12 French (0.33 mm: 1 French), usually from 3 French to 9 French. Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase qualities such as rotational strength, column strength, toughness, or pushability. Suitable catheter bodies may be formed by extrusion, with one or more channels being provided when desired. The catheter diameter can be modified by heat expansion and shrinkage using conventional techniques. The resulting catheters will thus be suitable for introduction into the vascular system, often the coronary arteries, by conventional techniques.

As noted, in certain aspects, the catheter body may be reinforced for torsional rigidity to increase axial torque transmission from the proximal to distal portion of the body. Torsional rigidity may be augmented through a variety of torsion members including wires, spines, shafts, braided or coiled materials, or a combination thereof. These members may be disposed around, on, or within some portion of the catheter body. Various members for increasing torsional rigidity are presented. An axial torque transmitting shaft may be an extruded single lumen, an extruded dual lumen, or an extruded single lumen with two shafts running through it. These lumens may be free floating or fixed between the proximal and distal ends of the catheter body but, in most embodiments, should be fixed to one or more of the guidewire lumens at the distal portion of the body. Fixation may be through heat fusion, adhesive, or other means known in the art. In certain embodiments, the axial torque transferring mechanism may include a separate lumen with a shaft run therethrough (example #1). The separate lumen, additional to the dual guidewire lumens, may run the length of the catheter body and may be fixed to the catheter body at least at the distal portion near the first and second exit ports and the imaging apparatus. The separate lumen should form a tight fit over the shaft to resist axial movement of the shaft relative to the separate lumen. The shaft can act as a spine to transfer axial torque and may be constructed of a variety of materials including metal, fiber, composite, and plastics or other polymers.

In certain aspects, the catheter may include a shaft made of a braided or coiled material (example #2), where the braided or coiled material is terminated at the distal and proximal ends in circumferential bands. The shaft may be terminated by coupling the cut braids at both proximal and distal ends with small bands or reducing the pitch of a coil at both ends until the coils are substantially touching. The shaft may be coupled to one or both of the guidewire lumens or otherwise joined to the catheter body at least at the distal portion. A torsion shaft may be incorporated in the construction of the catheter. In certain aspects, the inner diameter of the catheter body may be lined with a polymeric liner and the entire assembly may be reflowed to integrate the shaft into the catheter body. In some embodiments, the small bands coupling the cut braids at the distal and proximal ends of the shaft may be constructed of a polymer and can provide a surface which is easier to bond to the catheter body during manufacturing.

In some instances, catheters may include a hypotube inserted into a guidewire or microcable lumen (example #3). The hypotube may, like the separate lumen in example #1, tightly fit around a shaft and be fixed to the catheter body, at least at the distal portion, to provide a spine like support. In certain aspects, a third lumen may be constructed into the distal portion of the catheter while the proximal portion comprises two lumens (example #4). The third lumen may provide additional torsional rigidity at the distal portion of the catheter and may tightly contain a shaft as described in example #1. In some embodiments, a braided shaft constructed from, for example, a polymer, may be inserted into a compatible polymer jacket and fused with heat or by chemical process to the RX lumen or the distal portion of the OTW lumen (example #5).

The distal portion of the body, an imaging apparatus support, and/or a tracking tip include a pattern of markings positioned to show orientation of the distal portion of the body, an imaging apparatus support, and/or a tracking tip and to aid in navigation of the catheter and/or treatment delivery. Markings may be radiopaque so that they are observable from outside of the body using x-rays. Markers may be embedded inside the body of the device, and can be dimensioned to be compatible with various monitoring software such as software for correlating a single view from IVUS and angiogram images (e.g., SyncVision™, Volcano Corporation, San Diego, Calif.).

To aid in visualization and orientation of the distal portion of the body and the exit ports within vasculature, the distal portion of the body may include a pattern of radiopaque or other markers which may be externally monitored via, for example, x-ray.

In certain embodiments, catheters of the present disclosure may include one or more centering mechanisms disposed on the catheter body, catheter tip, or the imaging apparatus support. Centering mechanisms may be disposed at any suitable location along the length of the catheter body. In preferred embodiments, centering mechanisms are disposed near the distal end of the catheter and/or the first and/or second exit ports so that the first and/or second exit ports may be centered within a vessel by the centering mechanism. Centering mechanisms may include, for example, inflatable balloons, or collapsible structures such as a sheathable nitinol basket or other structure comprising a shape memory material. Centering mechanisms may have an unexpanded state in which they remain close to the catheter body and an expanded state wherein the centering mechanism expands radially from the surface of the catheter body in order to interact with a lumen wall to center the first and/or second exit ports within a cross-section of a vessel, artery, or other body lumen. A balloon centering mechanism may transition between an unexpanded and expanded state through application of a fluid or gas to inflate the one or more balloons. The catheter body may include an air or fluid line connecting the balloon centering mechanism to an air or fluid source. A pump may be used to force air or fluid into the centering balloon in order to expand it. Balloon centering mechanisms may be of any suitable shape or size.

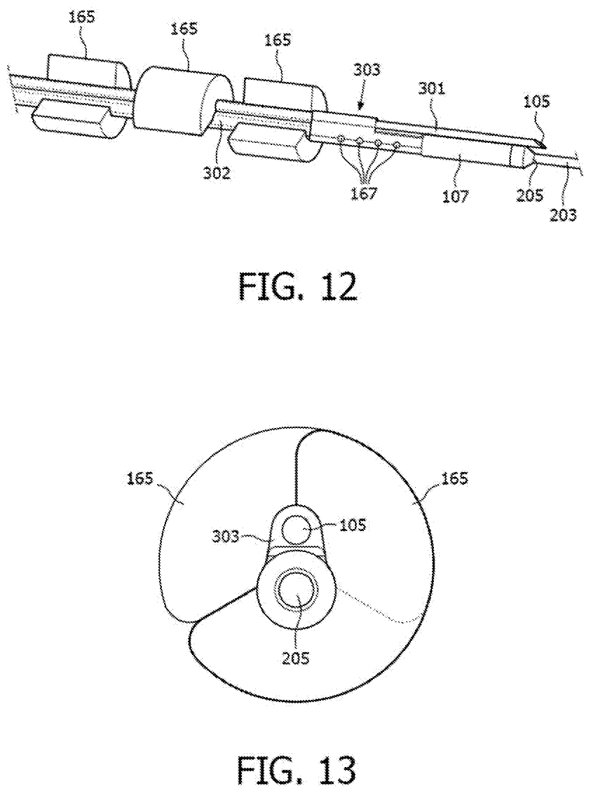

FIG. 12 shows an exemplary catheter configuration wherein three separate centering mechanisms 165 are spaced apart along the catheter body near an imaging apparatus support 303. The imaging apparatus support 303 comprises an imaging apparatus 107, a plurality of perfusion holes 167, a first guidewire lumen 301 having a first exit port 105 and a second guidewire lumen 302 having a second exit port 205 and containing a second guidewire 203. The centering mechanisms 165 are balloons which, when inflated, comprise a C-shaped cross section and surround a portion of the circumference of the catheter body. The three centering mechanisms 165 are positioned relative to each other so that the gaps in the C-shaped cross sections are offset from each other along the circumference of the catheter cross section as shown in FIG. 13. By offsetting the gaps, the balloon catheters provide a centering force to the catheter against a lumen wall around the entire circumference of the catheter surface while maintaining an open flow path for blood or other fluids within the body lumen. This may allow the centering mechanism to be used during treatment without disrupting blood flow within the lumen being treated, thereby avoiding problems caused by lack of blood flow to tissues and enabling sensors on the catheter to accurately track pressure or flow within the lumen in order, for instance, to determine effectiveness of a treatment such as removal of an occlusion. Balloon centering mechanisms may be placed in offset of one another along the device anywhere proximal first exit port 105 on the distal end of the catheter. Balloon centering mechanisms may be segmented circles with open sections to allow blood flow through. A helical open section orientation between multiple balloons may optimize centering efficiency, and blood flow rate. A profile view of the catheter shows that these balloons should center the device from 360° around the circumference of the catheter body. Multiple centering mechanisms 165 as shown in FIGS. 12 and 13, may allow for individual inflation or expansion so that only those centering mechanisms that are necessary need be deployed. In certain embodiments, multiple centering mechanisms 165 may be have a variety of sizes and shapes so that one or more centering mechanisms 165 may be selectively expanded based on the size and shape of the body lumen in which they are being deployed.

FIG. 15 shows a spiral-shaped balloon centering mechanism 165 which spirals around the circumference of the catheter body near the distal end of the catheter and the imaging apparatus support 303, proximal to the imaging apparatus 107 and the first 105 and second 205 exit ports. The imaging apparatus support 303 comprises an imaging apparatus 107, a plurality of perfusion holes 167, a first guidewire lumen 301 having a first exit port 105 and a second guidewire lumen 302 having a second exit port 205 and containing a second guidewire 203. The spiral-shaped centering mechanism 165 may enable greater flexibility of the catheter body than other designs, particularly when expanded. The spiral-shaped centering mechanism 165 provides centering force around the entire circumference of the outer catheter surface while maintaining an open flow path for blood or other fluids within the lumen.

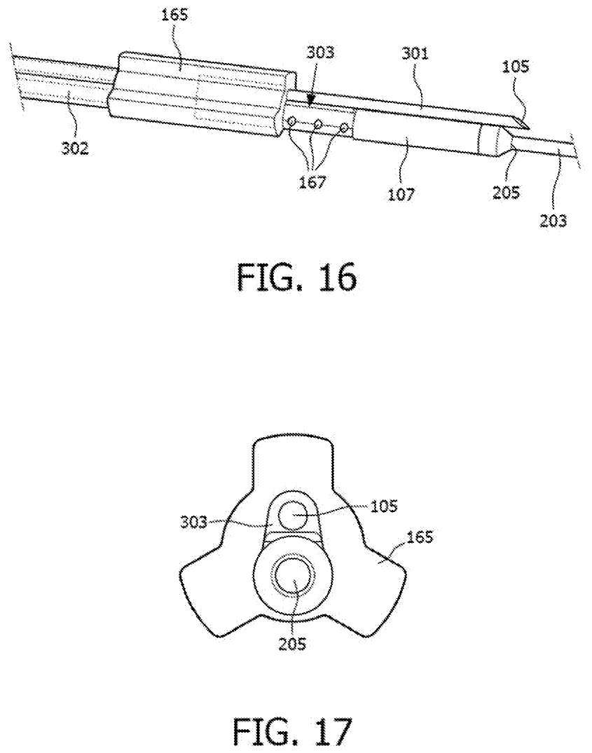

FIGS. 16 and 17 show a centering mechanism 165 comprising a balloon placed immediately proximal of the imaging apparatus support 303, the imaging apparatus 107, and the first 105 and second 205 exit ports, allowing the first (not shown) or second 203 guidewire to exit in the center of the centering mechanism 165 while preventing any damage thereto. The imaging apparatus support 303 comprises an imaging apparatus 107, a plurality of perfusion holes 167, a first guidewire lumen 301 having a first exit port 105 and a second guidewire lumen 302 having a second exit port 205 and containing a second guidewire 203. Disposing the centering mechanism 165 near the first 105 or second 205 exit port may provide more effective centering of those ports than where the centering mechanism 165 is disposed at a distance. The centering mechanism 165 shown in FIGS. 16 and 17 is a single segmented circle balloon with multiple open sections. A single segmented circle may provide circumferential centering force for the catheter against the wall of the body lumen while maintaining a blood or fluid flow path through the multiple open sections.

In various embodiments, a centering mechanism may comprise a collapsible structure such as a nitinol basket wherein a sheath maintains the mechanism in a collapsed, unexpanded state close to the catheter body and, when the sheath is removed, the mechanism expands. A sheath may be coupled to a release mechanism so that it may be manipulated from the proximal end of the catheter. In certain aspects, the sheath may be configured to be removed and replaced so that the centering mechanism may be collapsed after treatment for ease of removal from the vasculature.

In certain embodiments, a catheter with a centering mechanism may be advanced through the vasculature to a desired treatment location at which point the centering mechanism may be expanded or deployed in order to center the catheter and/or one or more exit ports thereof within the vasculature. Treatment may then be applied and the centering mechanism may be collapsed before removal of the catheter from the vasculature.

Dual Lumen Transducer Support

In certain embodiments, the distal portion of a catheter body may comprise an imaging apparatus, or transducer, support configured to house the imaging apparatus and the first and/or second exit ports. The transducer support may include an integrated tip or may be coupleable to a variety of interchangeable tips which may be selected based on the application. The transducer support may include features such as glue port holes to aid in catheter construction and/or functional measurement sensors for parameters such as pressure, flow, and velocity and may comprise, for example, optical sensors, microfabricated microelectromechanical (MEMS) pressure sensors, or ultrasound transducers, including Doppler velocity sensors, to measure the parameters.

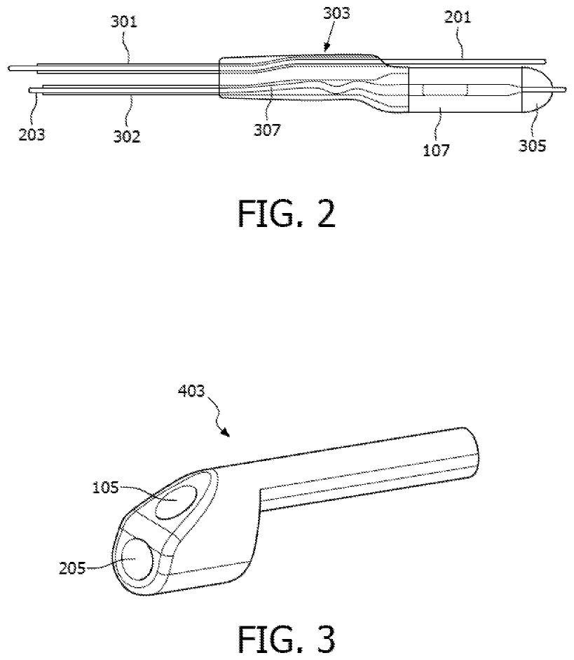

FIG. 2 shows an imaging apparatus support 303 with a first guidewire lumen 301 of the OTW type containing a first guidewire 201. The imaging apparatus support 303 includes an imaging apparatus 107 with a separate, short, single lumen tip 305 and a RX-type second guidewire lumen 302 disposed through the imaging apparatus 107 and the tip 305 and exiting therethrough. A second guidewire 203 is disposed within the second guidewire lumen 302 along with a microcable 307 connected to the imaging apparatus 107.

In some aspects, the transducer support may be rigid in order to maintain a relative orientation between the first and second exit ports, the imaging apparatus, and in some instances a functional measurement sensor. In most embodiments the imaging apparatus support may have a diameter that generally matches the proximal portion of the catheter body, however, in other embodiments, the distal portion may be larger or smaller than the proximal portion of the catheter. The imaging apparatus support can be formed from materials that are rigid or which have very low flexibilities, such as metals, hard plastics, composite materials, NiTi, steel with a coating such as titanium nitride, tantalum, ME-92 (antibacterial coating material), or diamonds. Most usually, the distal end of the catheter body will be formed from stainless steel or platinum/iridium.

Imaging apparatus supports and/or tracking tips may be constructed with one or more lumens and may be extruded from a raw material or additive manufactured using, for instance, 3D printing techniques. Imaging apparatus supports and/or tracking tips may also be made through molding casting or other suitable construction techniques known in the art and suited to the material from which the component is constructed. FIG. 7 shows a cutaway view of an imaging apparatus support 303 including a first exit port 105 offset from a second exit port 205, and also including a glue port 501 to aid in catheter construction. Imaging apparatus supports and/or tracking tips may include a step 607 in an inner lumen. The inner lumen may have a greater diameter than the first or second guidewire lumen proximal to the step 607 and may have a lesser diameter than the first or second guidewire lumen distal to the step 607. In certain embodiments, the inner lumen of the imaging apparatus support or a tracking tip may taper, narrowing toward their distal ends or toward steps 607. The inner lumen and/or step 607 of an imaging apparatus support or tracking tip may help during construction of the catheter by centering the guidewire lumen as it is inserted into the imaging apparatus support or tracking tip and providing a stop to indicate full insertion. The imaging apparatus support or tracking tip may also include one or more glue ports 501 through which an adhesive may be introduced to secure a guidewire lumen to the imaging apparatus support or tracking tip after the guidewire lumen has been inserted therein. An imaging apparatus support or tracking tip may include a single proximal inner lumen 609 which separates into multiple distal inner lumens. The separate distal inner lumens may provide a localized joint just proximal to the imaging apparatus and/or exit ports that force both guidewire lumens to realign parallel to one another. Parallel lumens near the imaging apparatus and/or the exit ports may increase the centering strength of the RX guidewire when delivering treatment through the OTW lumen or vice versa.

In certain instances, the imaging apparatus support may be integrally formed with the catheter body and/or a single or dual lumen tip. In embodiments with a dual lumen tip, the exit port for one of the lumens can be disposed on the imaging apparatus support or the distal portion of the catheter body. In preferred embodiments, the first and second exit ports are disposed near the imaging apparatus and, in embodiments including an imaging apparatus support, the support. The first exit port, the second exit port, or both, may be located within 1 cm, 2 cm, 3 cm, 4 cm, 5 cm, 6 cm, 7 cm, 8 cm, 9 cm, or 10 cm of the imaging apparatus or imaging apparatus support. The first exit port may be disposed on the catheter distal to or proximal to the imaging apparatus support or the imaging apparatus. The second exit port may be disposed on the catheter distal to or proximal to the imaging apparatus support or the imaging apparatus. The two exit ports may or may not be disposed on the same side of the imaging apparatus.

Exit ports may be disposed at the same location along the catheter, imaging apparatus support, or dual lumen tracking tip, or may be offset or disposed on separate components on the catheter body. FIGS. 5-9 illustrate various embodiments of imaging apparatus supports. FIG. 5 shows a dual lumen imaging apparatus support 303 with a first guidewire lumen 301 and a second guidewire lumen 302 with a first guidewire 201 and a second guidewire 203 disposed therein. The imaging apparatus support 303 includes an imaging apparatus 107 with the second guidewire 203 and guidewire lumen 302 passing therethrough. The first exit port 105 is offset from the second exit port 205 and is proximal to the imaging apparatus 107 while the second exit port is distal to the imaging apparatus 107. The imaging apparatus support 303 further comprises a functional measurement sensor 401.

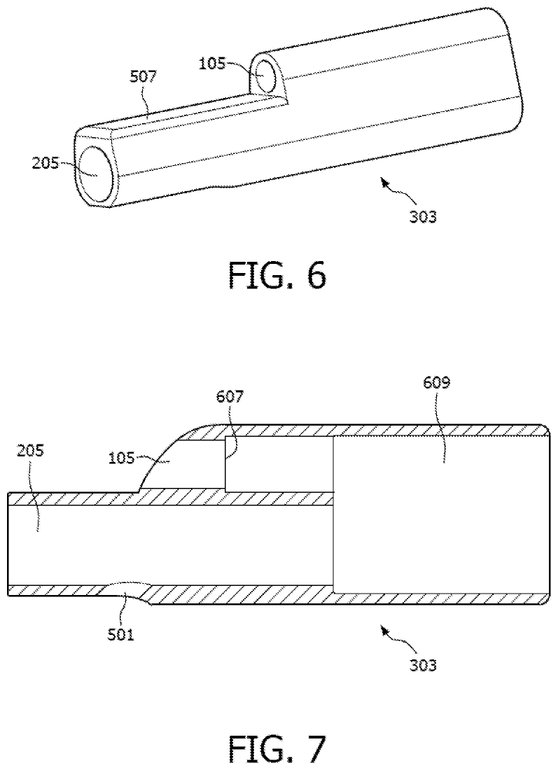

FIG. 6 shows a dual lumen imaging apparatus support 303 with a first exit port 105 that is offset from and proximal to the second exit port 205. Additionally, the surface of the imaging apparatus support 303 after the first exit port 105 is flattened 507. The imaging apparatus support 303 further comprises a functional measurement sensor 401. In certain embodiments where an exit port is located proximal to the end of the catheter, tip, or transducer support, the surface of the catheter, tip, or transducer support distal to the exit port may be flattened as in FIG. 6. The flat surface 507, may provide structural support against guidewire or catheter kinking and/or may provide a mounting point for the transducer or imaging apparatus. An imaging apparatus may be mounted using, for example, a flex leg and adhesive or mechanical fasteners. A flattened mounting surface 507 may provide additional space for padding. Padding at the imaging apparatus mounting surface can mitigate the risk of physically induced image failure.

FIG. 7 illustrates an imaging apparatus support 303 with a first exit port 105 that is offset from and proximal to the second exit port 205. Additionally, the surface of the imaging apparatus support 303 after the first exit port 105 is flattened 507. The imaging apparatus support 303 further comprises a functional measurement sensor 401.

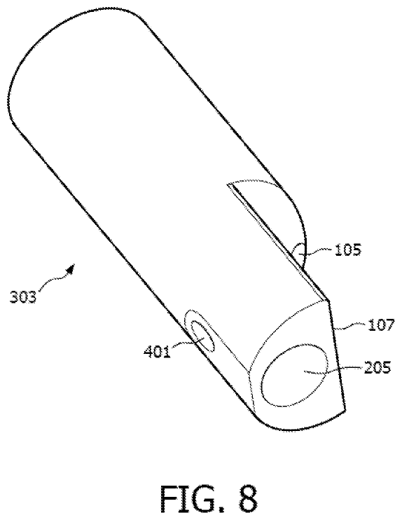

Exit ports of the present disclosure may be flat or perpendicular to the guidewire lumen which they terminate as illustrated by the second exit port 205 in FIG. 8. Exit ports may alternatively be rounded as shown by the first exit port 105 in FIG. 8 or angled relative to the catheter, guidewire lumens, tracking tip, or imaging apparatus support as shown by the first exit port 105 in FIG. 7. Angled or rounded exit ports may ease passage of the catheter through a body lumen. Exit ports may form an obtuse angle with a line tangential to the distal portion of the elongated body, imaging apparatus support, or tracking tip.

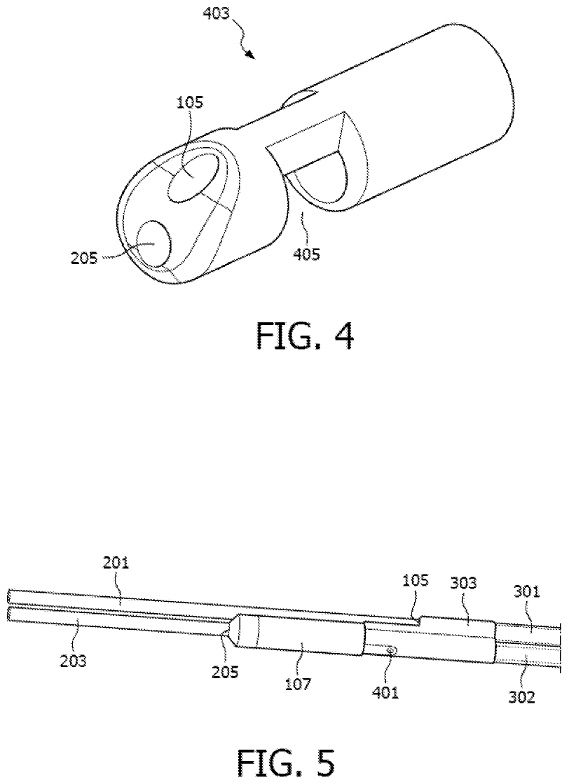

Exemplary embodiments of separate, dual lumen tracking tips 403 are shown in FIGS. 3 and 4. FIG. 3 shows a dual lumen tracking tip 403 with a first exit port 105 and a second exit port 205 disposed thereon. The first exit port 105 is angled relative to the second exit port 205 so that the tip presents a frontal surface with reduced drag. FIG. 4 shows a dual lumen tracking tip 403 with a first exit port 105 and a second exit port 205 disposed thereon with a rounded frontal surface area. The dual lumen tracking tip 403 is configured to fully encase the imaging apparatus and includes a cutout 405 for the imaging plane of the imaging apparatus so that the tracking tip does not interfere with the intralumenal imaging. In various embodiments, dual lumen tracking tips may be used in conjunction with a dual lumen imaging apparatus support or an imaging apparatus may be coupled directly proximal or distal to the dual lumen tracking tip.

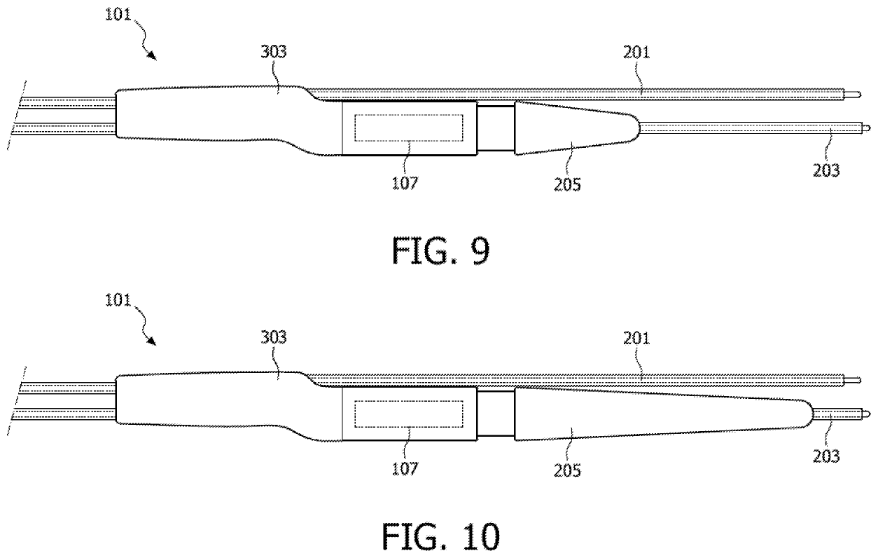

Examples of dual lumen catheters are shown in FIGS. 9 and 10. FIG. 9 illustrates a dual lumen catheter 101 with an imaging apparatus 107 housed in an imaging apparatus support 303 and a separate tip 205. The second guidewire 203 travels through the imaging apparatus 107 and exits distally to the first guidewire 201. FIG. 10 illustrates a dual lumen catheter 101 with an imaging apparatus 107 housed in an imaging apparatus support 303 and an extended, integrated tip 205. The second guidewire 203 travels through the imaging apparatus 107 and exits distally to the first guidewire 201.

The proximal portion of the catheter may terminate at a hub such as a Y-arm, with, for instance, entry ports for the first and second catheter. A microcable coupled to the imaging apparatus at the distal portion of the catheter may emerge from a dedicated or shared purpose lumen at the proximal end of the catheter and may be coupled to a computer, a monitoring system, or other equipment configured to interpret and convey information from the imaging apparatus.

In certain aspects, the imaging apparatus may be coupled, through a microcable or otherwise, to a controller including a processor, or to a processor, to control and/or record data from the imaging apparatus. The controller will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of catheter system and within the controller.

In certain embodiments, the controller may direct rotational or longitudinal movement of the imaging apparatus on the catheter body or on a drive cable. The controller can be configured to receive and display imaging data from the imaging apparatus and to coordinate intraluminal movements of the imaging apparatus while receiving data (e.g., in pull-back IVUS or pull-back OCT). Furthermore, the controller may also control movement and activation of the denervation assembly to facilitate placement of the denervation assembly in relation to the target tissue and delivery of denervation therapy to the target tissue. In certain embodiments, the controller may control deployment of an expandable member in order to bring a denervation assembly mounted thereon into contact with target tissue on the wall of the lumen (e.g., renal denervation in a renal artery).

In other embodiments, the imaging apparatus may rotate or translate using drive cables within the catheter body. Catheters having imaging assemblies that rotate and translate are known generally as “pull-back” catheters. The principles of pull-back OCT are described in detail in U.S. Pat. No. 7,813,609 and US Patent Publication No. 20090043191, both of which are incorporated herein by reference in their entireties. The mechanical components, including drive shafts, rotating interfaces, windows, and couplings, are similar between the various forms of pull-back imaging.

In various embodiments, the imaging apparatus may be integrated within the body of the catheter, may circumscribe the catheter, may be placed on a distal end face of the catheter, and/or may run along the body of the catheter. The catheter may also include an outer support structure or coating surrounding the imaging apparatus.

The guidewire lumens of the dual lumen imaging apparatus may be fixed relative to each other and the imaging apparatus. Alternatively, one or more of the guidewire lumens may be moveable relative to the imaging apparatus, each other, or both so that the relative position of the first and/or second exit ports to the imaging apparatus may be altered by extending or retracting a guidewire lumen out of or into the catheter body.

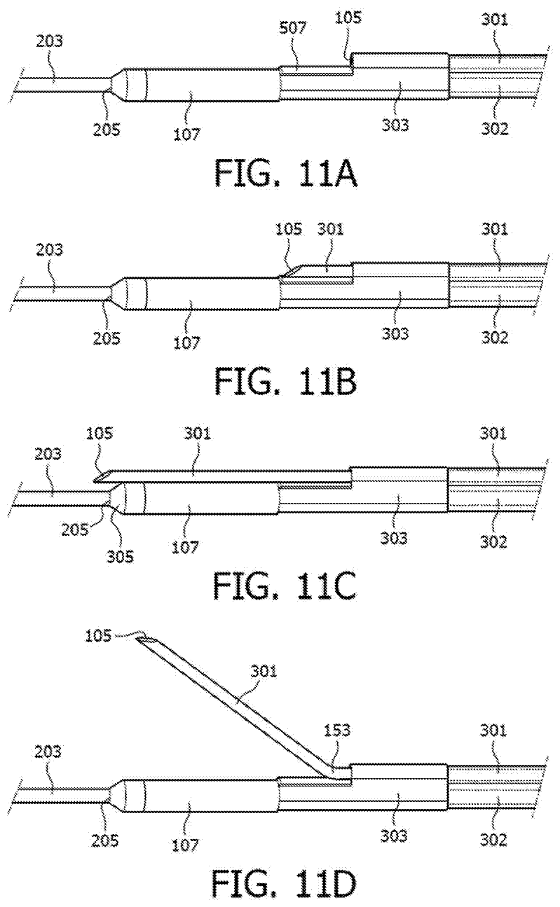

In certain aspects, the first guidewire lumen may comprise a spring loaded needle forming an OTW lumen. The spring loaded needle, which has a lumen, may be constructed from a material such as stainless steel or nitinol. The spring loaded needle may be controllable from the proximal end of the device. Any of the first exit port 105 configurations shown in FIGS. 11A-11D may be incorporated independently or in combination into a dual lumen imaging apparatus. For example, a spring loaded first guidewire lumen or needle 301 may be movable in relation to a dual lumen imaging apparatus support 303 so that the position of the first exit port 105 may be varied in relation to the imaging apparatus 107 by advancing or withdrawing the first guidewire lumen or needle 301 with respect to the imaging apparatus support 303. Where the first guidewire lumen or needle 301 comprises a needle lumen, the first exit port 105 may be sharp and/or beveled to allow insertion into tissue or occlusive material.

In certain embodiments, the entire needle lumen or sections of it may be uniformly or variably laser cut or braided to improve flexibility and ease advancement of the catheter through a body lumen. In certain embodiments, the first guidewire lumen or needle 301 may include a pre-bent portion 153 which may be accomplished, for example, through use of a shape-memory material such as nitinol. A pre-bent portion 153 may comprise a variety of angles such as less than 1 degree, or 1, 5, 10, 15, 20, 25, 30, 35, 40, 45, 50, 55, 60, 70, 80, 90, or more degrees. In preferred embodiments, the pre-bent portion 153 comprises an angle of 90 degrees or less to ease retraction of the first guidewire lumen or needle into the imaging apparatus support 303 after use and before withdraw of the catheter from the body lumen.

In various embodiments, the catheter body may comprise a material with greater rigidity than the first guidewire lumen or needle 301 so that, when withdrawn into the imaging apparatus support 303 and catheter body, the first guidewire lumen or needle remains approximately parallel to the catheter body and the second guidewire lumen 303 as shown in FIGS. 11A-11C; but when the first guidewire lumen or needle 301 is extended so that the pre-bent portion 153 is beyond the imaging apparatus support 303, the first guidewire lumen or needle 301 assumes the angle of the pre-bent portion 153 as shown in FIG. 11D. The length of the guidewire lumen or needle 301 distal to the pre-bent portion 153 may be configured along with the angle of the pre-bent portion 153 in order to achieve a variety of orientations of the first exit port 105 relative to the imaging apparatus support 303. In certain aspects the first guidewire lumen or needle 301 may have multiple pre-bent portions 153 spaced at various lengths along the distal end of the first guidewire lumen or needle 301 so that the angle of the first guidewire lumen or needle 301 relative to the catheter body may be increased in steps by advancing one or more pre-bent portions 153 beyond the distal end of the imaging apparatus support 303. The use of multiple discrete pre-bent portions 153 may also be used to achieve a cumulative angle greater than 90 degrees without introducing problems with retraction of the first guidewire lumen or needle 301 into the into the imaging apparatus support 303 after use and before withdraw of the catheter from the body lumen. In certain embodiments the location of the first exit port 105 may be further modified through axial rotation of the first guidewire lumen or needle 301 after the pre-bent portion 153 has been extended beyond the distal opening of the imaging apparatus support 303.

FIG. 11A shows a dual lumen imaging apparatus support 303 with an imaging apparatus 107, the second guidewire lumen 302 passing therethrough. An RX guidewire 203 emerges from the second exit port 205, distal to the imaging apparatus 107. The first guidewire lumen or needle 301 comprises a first exit port 105 that is disposed proximal to the imaging apparatus 303 by a distance. The surface of the imaging apparatus support 303 after the first exit port 105 is flattened 507.

FIG. 11B shows a dual lumen imaging apparatus support 303 with an imaging apparatus 107, second guidewire lumen 302 passing therethrough. An RX guidewire 203 emerges from the second exit port 205, distal to the imaging apparatus 107. The first guidewire lumen or needle 301 comprises a first exit port 105 that is disposed at the proximal edge of the imaging apparatus 303.

FIG. 11C shows a dual lumen imaging apparatus support 303 with an imaging apparatus 107, second guidewire lumen 302 passing therethrough. An RX guidewire 203 emerges from the second exit port 205, distal to the imaging apparatus 107. The first guidewire lumen or needle 301 comprises a first exit port 105 that is disposed distal to the imaging apparatus 107 and at or just distal to the tip 305 and the second exit port 205.

FIG. 11D shows a dual lumen imaging apparatus support 303 with an imaging apparatus 107, second guidewire lumen 302 passing therethrough. An RX guidewire 203 emerges from the second exit port 205, distal to the imaging apparatus 107. The first guidewire lumen or needle 301 is angled from the imaging apparatus 107 by a pre-bent portion 153.

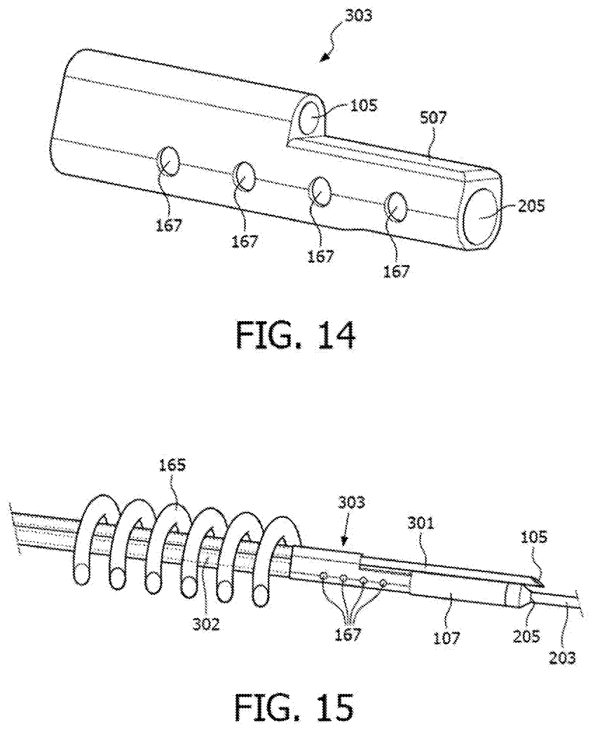

In certain embodiments, a device of the present disclosure may include one or more perfusion holes disposed along the device. Perfusion holes may be disposed along the OTW lumen. Perfusion holes can be perpendicular to the OTW lumen or angled. Perfusion holes 167 may be disposed on the catheter tip, along the catheter body or on a dual lumen imaging apparatus support 303 as shown in FIG. 12 or 14-16. Perfusion holes 167 may be disposed proximal to the imaging apparatus 107 and the first 105 and second 205 exit ports and may be disposed on one side of the catheter of imaging apparatus support 303 or may be disposed on multiple sides along the outer surface of the catheter of imaging apparatus support 303.

Imaging Apparatus

In certain embodiments, the imaging and treatment device of the present disclosure includes an imaging apparatus. The imaging apparatus may be disposed on the catheter body, an imaging apparatus support at the distal end of a catheter body, or on a drive cable depending on the imaging technology being employed. Any imaging apparatus may be used with devices and methods of the present disclosure, such as optical-acoustic imaging apparatus, intravascular ultrasound (IVUS) or optical coherence tomography (OCT). The imaging apparatus is used to send and receive signals to and from the imaging surface that form the imaging data.

In some embodiments, the imaging apparatus is an IVUS imaging apparatus. The imaging apparatus can be a phased-array IVUS imaging apparatus, a pull-back type IVUS imaging apparatus, including rotational IVUS imaging assemblies, or an IVUS imaging apparatus that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein in their entirety.

IVUS imaging is widely used as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer assembly is located at the tip of a flexible driveshaft that spins inside a plastic sheath inserted into the vessel of interest. The transducer assembly is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include, for example the REVOLUTION 45 MHz catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. Pat. No. 6,200,268), the content of each of which is incorporated by reference herein in its entirety. In flow imaging mode, the transducer elements are operated in a different way to collect the information on the motion or flow. This process allows one image (or frame) of flow data to be acquired. The particular methods and processes for acquiring different types of intravascular data, including operation of the transducer elements in the different modes (e.g., gray-scale imaging mode, flow imaging mode, etc.) consistent with the present disclosure are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is ChromaFlo® (IVUS fluid flow display software offered by the Volcano Corporation). Suitable phased array imaging assemblies are found on Volcano Corporation's EAGLE EYE Platinum Catheter, EAGLE EYE Platinum Short-Tip Catheter, and EAGLEEYE Gold Catheter. Catheters and imaging apparatuses of the present disclosure may be compatible with automated body lumen measurement software such as VH® IVUS (Volcano Corporation, San Diego, Calif.), image highlighting software for blood, plague and software for correlating a single view from IVUS and angiogram images such as SyncVision™ (Volcano Corporation, San Diego, Calif.).

In addition to IVUS, other intraluminal imaging technologies may be suitable for use in methods of the present disclosure for assessing and characterizing vascular access sites in order to diagnose a condition and determine appropriate treatment. For example, an Optical Coherence Tomography (OCT) catheter may be used to obtain intraluminal images in accordance with the present disclosure. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the present disclosure may obtain imaging data from an OCT system, including OCT systems that operate in either the time domain or frequency (high definition) domain. Basic differences between time-domain OCT and frequency-domain OCT is that in time-domain OCT, the scanning mechanism is a movable mirror, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference mirror, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has allowed the determination of distance and scattering intensity of multiple scatters lying along the illumination axis by analyzing the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. Nos. 7,999,938; 7,995,210; and 7,787,127 and differential beam path systems are described in U.S. Pat. Nos. 7,783,337; 6,134,003; and 6,421,164, the contents of each of which are incorporated by reference herein in its entirety.

In certain embodiments, angiogram image data is obtained simultaneously with the imaging data obtained from the imaging apparatus and/or imaging guidewire of the present disclosure. In such embodiments, the catheter and/or guidewire may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

In certain embodiments, the imaging apparatus may be an optical-acoustic imaging apparatus. Optical-acoustic imaging apparatuses include at least one imaging element to send and receive imaging signals. In one embodiment, the imaging apparatus includes at least one acoustic-to-optical transducer. In certain embodiments, the acoustic-to-optical transducer is a Fiber Bragg Grating within an optical fiber. In addition, the imaging assemblies may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging assemblies suitable for use in devices of the present disclosure are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interference in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

In certain embodiments, the imaging apparatus includes a piezoelectric element to generate the acoustic or ultrasound energy. In such aspect, the optical fiber of the imaging apparatus may by coated by the piezoelectric element. The piezoelectric element may include any suitable piezoelectric or piezoceramic material. In one embodiment, the piezoelectric element is a poled polyvinylidene fluoride or polyvinylidene difluoride material. The piezoelectric element can be connected to one or more electrodes that are connected to a generator that transmits pulses of electricity to the electrodes. The electric pulses cause mechanical oscillations in the piezoelectric element, which generates an acoustic signal. Thus, the piezoelectric element is an electric-to-acoustic transducer. Primary and reflected pulses (i.e. reflected from the imaging medium) are received by the Bragg Grating element and transmitted to an electronic instrument to generate an imaging.

In some embodiments, the imaging apparatus includes an optical fiber with Fiber Bragg Grating and a piezoelectric element. In this embodiment, an electrical generator stimulates the piezoelectric element (electrical-to-acoustic transducer) to transmit ultrasound impulses to both the Fiber Bragg Grating and the outer medium in which the device is located. For example, the outer medium may include blood when imaging a vessel. Primary and reflected impulses are received by the Fiber Bragg Grating (acting as an acoustic-to-optical transducer). The mechanical impulses deform the Bragg Grating and cause the Fiber Bragg Grating to modulate the light reflected within the optical fiber, which generates an interference signal. The interference signal is recorded by electronic detection instrument, using conventional methods. The electronic instrument may include a photodetector and an oscilloscope. An image can be generated from these recorded signals. The electronic instruments modulation of light reflected backwards from the optical fiber due to mechanical deformations. The optical fiber with a Bragg Grating described herein, the imaging apparatus described herein and other varying embodiments are described in more detail in U.S. Pat. Nos. 6,659,957 and 7,527,594 and in U.S. Patent Publication No. 2008/0119739.

In another aspect, the imaging apparatus does not require an electrical-to-acoustic transducer to generate acoustic/ultrasound signals. Instead, the imaging apparatus utilizes the one or more Fiber Bragg Grating elements of the optical fiber in combination with an optical-to-acoustic transducer material to generate acoustic energy from optical energy. In this aspect, the acoustic-to-optical transducer (signal receiver) also acts as an optical-to-acoustic transducer (signal generator).

To generate the acoustic energy, imaging apparatus may include a combination of blazed and unblazed Fiber Bragg Gratings. Unblazed Bragg Gratings typically include impressed index changes that are substantially perpendicular to the longitudinal axis of the fiber core of the optical fiber. Unblazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber. Blazed Bragg Gratings reflect optical energy away from the longitudinal axis of the optical fiber.

One or more imaging assemblies may be incorporated into an imaging guidewire or the catheter to allow an operator to image a luminal surface. The one or more imaging assemblies of the imaging guidewire or catheter are referred to generally as an imaging apparatus. In some embodiments, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

In some aspects, the transducers may comprise capacitive micromachined ultrasonic transducers (CMUTs). CMUTs, which uses micromachining technology, allows for miniaturize device dimensions and produces capacitive transducers that perform comparably to the piezoelectric counterparts. CMUTs are essentially capacitors with one moveable electrode. If an alternating voltage is applied to the device then the moveable electrode begins to vibrate, thus causing ultrasound to be generated. If the CMUTs are used as receivers, then changes in pressure such as those from an ultrasonic wave cause the moveable electrode to deflect and hence produce a measurable change in capacitance. CMUT arrays can be made in any arbitrary geometry with very small dimensions using photolithographic techniques and standard microfabrication processes.

In some aspects, the transducers may comprise piezoelectric micromachined ultrasonic transducers (pMUTs), which are based on the flexural motion of a thin membrane coupled with a thin piezoelectric film. It should be noted that pMUTs exhibit superior bandwidth and offer considerable design flexibility, which allows for operation frequency and acoustic impedance to be tailored for numerous applications.

Methods

Catheters of the present disclosure may be used to access various healthy and diseased body lumens and, in particular, lumens of the vasculature. The real-time images obtained may be used to locate a region or location of interest within a body lumen and to guide and observe the delivery and after-effect of various treatments. Regions of interest are typical regions that include a defect or tissues requiring treatment. The devices and methods, however, are also suitable for treating stenosis of body lumens and other hyperplastic and neoplastic conditions in other body lumens, such as the ureter, the biliary duct, respiratory passages, the pancreatic duct, the lymphatic duct, and the like. In addition, the region of interest can include, for example, a location for stent placement or a location including plaque or diseased tissue that needs to be removed or treated. In some instances, the region of interest may include the renal artery where renal denervation therapy may be applied to the afferent and efferent nerves therein.

Catheters of the present disclosure may be used in combination with a variety of treatment methods to treat a variety of vascular problems. In certain aspects, the catheter may serve as a delivery catheter, ablation catheter, extraction catheter or energizing catheter to perform an intraluminal procedure. The catheter may include a denervation assembly to perform an intraluminal procedure. An OTW guidewire lumen may act as a utility lumen while an additional RX lumen acts as a delivery lumen, or vice versa. In some embodiments, methods can include treating a chronic total occlusion. In catheters including a functional measurement sensor, the sensor may be used in combination with or independent of the imaging apparatus to verify position of the distal portion of the body at the chronic total occlusion by, for example, sensing a change in pressure. The first guidewire for support while crossing the chronic total occlusion and a therapy may be delivered to the chronic total occlusion through the second guidewire lumen while imaging local to the first and second exit ports from the imaging apparatus informs the procedure.

During a procedure, the imaging apparatus may be used to image cross-sections of the luminal surface and to visualize the position of one or more exit ports. In addition, the catheter may also include forward or distal facing imaging assemblies to image the luminal space and/or any procedure in front of or distal to the catheter. For example, the imaging apparatus can axially image a luminal surface for the location and selection of a region of interest suspected of containing afferent and efferent nerves for the accurate and targeted delivery of a treatment. This greatly improves visualization during the procedure by allowing an operator to have real-time images of the vessel wall while the denervation assembly of the catheter is engaged with that portion of the vessel wall. After the treatment procedure, the imaging apparatus of the catheter can be used to perform a final visualization of the luminal surface before the catheter is removed from the patient.

The devices of the present disclosure may include static imaging assemblies that do not move with respect to the catheter body, or moving imaging assemblies. For example, the imaging apparatus may be a phased array of ultrasonic transducers for IVUS imaging, or a collection of CCD arrays. An array of assemblies will typically cover a circumference of the catheter to provide a 360° view of the lumen.

Catheters of the present disclosure may be used to deliver intravascular treatment. In certain embodiments, one of the guidewire lumens may be used for stability or to provide support while another guidewire is removed or advanced through the other guidewire lumen. For example, the catheter may reach, along a first guidewire in a first guidewire lumen, a bifurcation in the vasculature, observable via the imaging apparatus on the distal portion of the catheter. After observing the bifurcation and determining a desired route, a user may select a shaped guidewire, less rigid than the first guidewire, to insure access to the desired branch of the bifurcation. The shaped guidewire may by advanced through the second guidewire lumen while the first guidewire maintains support of the catheter, out of the second exit port and into the desired branch at which point the first guidewire be retracted slightly allowing the catheter to follow the shape of the second, shaped guidewire and the catheter can be advanced into the desired branch.

Other embodiments of catheters and systems of using them, not disclosed herein, will be evident to those of skill in the art, and are intended to be covered by the claims listed below.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the present disclosure and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this present disclosure in its various embodiments and equivalents thereof.

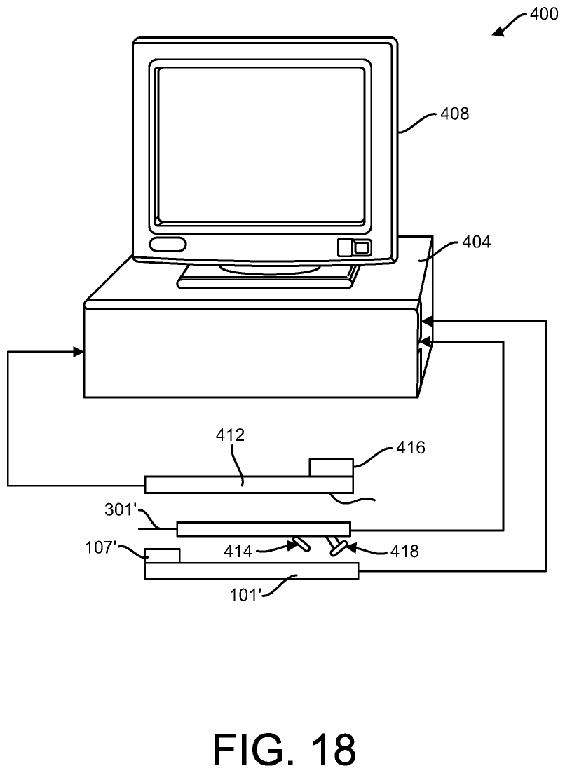

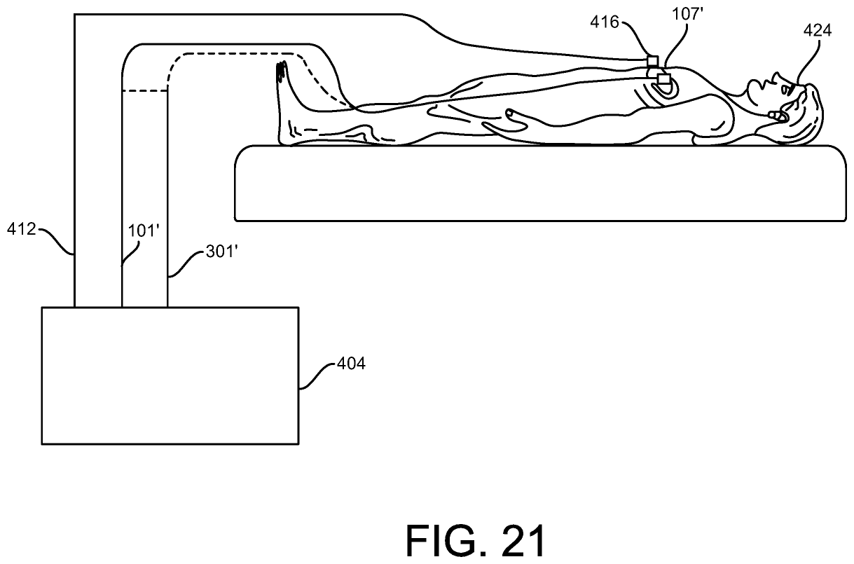

As discussed above, the present disclosure includes imaging and treating intravascular tissue structures. Referring to FIG. 18 and FIG. 21, there is shown a data-acquisition system, a patient monitoring system, and/or a therapeutic and control system 400 for imaging and treating soft tissue structures of a patient using one or more devices (or a variation thereof) discussed herein, wherein the soft tissue structure are located exterior of the vasculature. The data-acquisition system may acquire data, a patient monitoring system may acquire data and provide the data and/or additional information to the user, and/or a therapeutic and control system may perform all of the foregoing. The patient monitoring system 400 may include a monitoring system 404, which is electrically connected to a catheter 101′, which is similar to catheter 101 discussed herein above and used to acquire RF backscattered data from the vascular structure (e.g., a blood vessel, etc.). In lieu of or in addition to the catheter 101 having the ability to acquire RF backscattered data from the vascular structure in which the catheter 101′ is inserted, the catheter 101′ has the ability to acquire RF backscattered data from the soft tissue in the region or adjacent the vasculature. The present disclosure also contemplates using an externally applied imaging device, such as an ultrasound device 412 having a transducer 416. The externally applied ultrasound device 412 can be used in lieu of or in conjunction with the catheter 101′ and can be used to locate and identify the targeted soft tissue of interest. That is, referring to FIG. 21, both the externally applied ultrasound device 412 and/or catheter 101′ can be used to locate and identify the targeted soft tissue of interest, wherein the externally applied ultrasound device 412 identifies the targeted soft tissue from exterior the patient 424, and the catheter 101′ identifies the targeted soft tissue upon insertion of the catheter 101′ into the patient's vasculature. If both the externally applied ultrasound device 412 and the catheter 101′ are used, which may beneficial to increase the accuracy of identifying the location of the targeted soft tissue and aiding in delivery of the needle 301′, the ultrasound device 412 and the catheter 101′ can either be used serially or simultaneously.

The data-acquisition system, a patient monitoring system, and/or a therapeutic and control system 400 in conjunction with the ultrasound device 412 and the catheter 101′ have the ability to locate, determine the size, density and potentially the type of targeted tissue. Once the location, size, density and/or type of targeted tissue is identified using either one of or both of the transducers 107′, 416 with the monitoring system 404, the needle 301′ is accurately inserted into the vasculature of the patient. The needle 301′ is either inserted directly into the vasculature or through the catheter 101′, which includes the transducer 107′, or another type of catheter that may not have a transducer. After placing the needle 301′ within the desired location of the vasculature of the patient, the needle 301′ is extended beyond the catheter 101′, pierces and traverses the vascular structure 420, and enters into the soft tissue of the patient 424. Due to the accuracy of the one or more of the transducers 107′, 416, the location, size, density and/or type of target within the soft tissue is provided by the monitoring system 404 to the clinician via the display 408. The monitoring system 404 and/or clinician can use this information to accurately insert the port of the needle into the targeted soft tissue and deliver the precisely needed amount of therapeutic agent to the targeted soft tissue through the needle 301′.

The display 408 displays an image of the targeted tissue, along with the location, size and density of the targeted tissue, using a graphical user interface (GUI) (not shown) operating on the monitoring system 404. It should be appreciated that the monitoring system 404 or computing devices depicted herein (e.g., 404, etc.) includes, but is not limited to, personal computers, mainframe computers, PDAs, and all other computing devices, including medical (e.g., ultrasound devices, thermographic devices, optical devices, MRI devices, etc.) and non-medical devices. In that regard, the monitoring system 404 may be a passive monitoring system that displays images or an active, interactive or smart monitoring system or computing device which interprets data and provides the clinician's suggests to operate the needle 301′, the device 412 and the catheter 101′ or even aids the clinician in partially or automatically controlling the needle 301′, the device 412 and the catheter 101′.

According to various embodiments of the disclosed subject matter, any number of the components depicted in FIG. 18, including the monitoring system 404, the catheter 101′, the ultrasound device 412, and the needle catheter 301′ may be implemented on one or more computing devices.

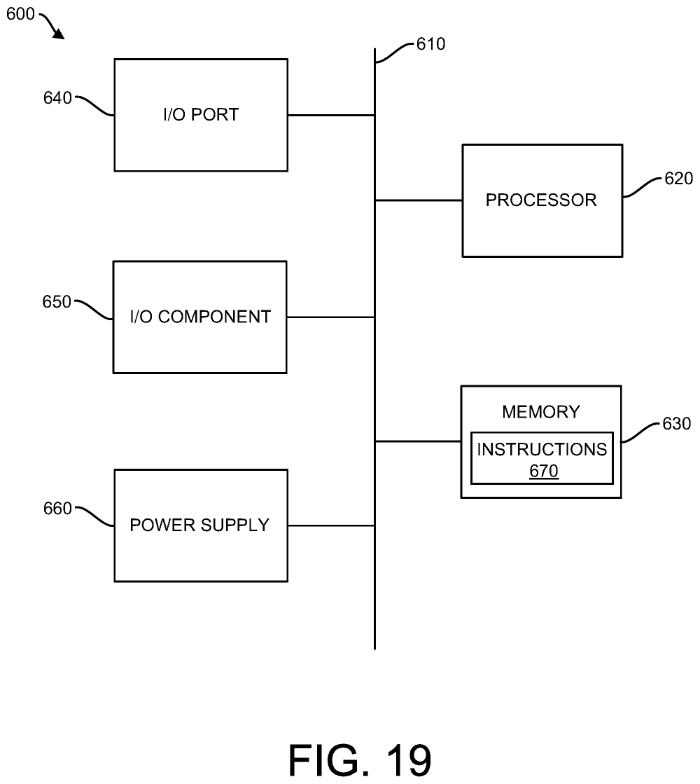

Referring to FIG. 19, there is shown a block diagram depicting an illustrative computing device 600, in accordance with various embodiments of the present disclosure. The computing device 600 may include any type of computing device suitable for implementing aspects of embodiments of the disclosed subject matter. Examples of computing devices include specialized computing devices 600 or general-purpose computing devices such “workstations,” “servers,” “laptops,” “desktops,” “tablet computers,” “hand-held devices,” “general-purpose graphics processing units (GPGPUs),” and the like, all of which are contemplated within the scope of this disclosure.

In embodiments, the computing device 600 includes a bus 610 that, directly and/or indirectly, couples the following devices: a processor 620, a memory 630, an input/output (I/O) port 640, an I/O component 650, and a power supply 660. Any number of additional components, different components, and/or combinations of components may also be included in the computing device 600. The I/O component 650 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The bus 610 represents what may be one or more busses (such as, for example, an address bus, data bus, or combination thereof). Similarly, in embodiments, the computing device 600 may include a number of processors 620, a number of memory components 630, a number of I/O ports 640, a number of I/O components 650, and/or a number of power supplies 660. Additionally any number of these components, or combinations thereof, may be distributed and/or duplicated across a number of computing devices.

In embodiments, the memory 630 includes computer-readable media in the form of volatile and/or nonvolatile memory and may be removable, non-removable, or a combination thereof. Computer-readable media is a storage and/or transmission medium that participate in providing instructions to a processor for execution. Media is commonly tangible and non-transient and can take many forms, including but not limited to, non-volatile media and volatile media and transmission media Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals. In embodiments, the memory 630 stores computer-executable instructions 670 for causing the processor 620 to implement aspects of embodiments of system components discussed herein and/or to perform aspects of embodiments of methods and procedures discussed herein.

The computer-executable instructions 670 may include, for example, computer code, machine-useable instructions, and the like such as, for example, program components capable of being executed by one or more processors 620 associated with the computing device 600. Program components may be programmed using any number of different programming environments, including various languages, development kits, frameworks, and/or the like. Some or all of the functionality contemplated herein may also, or alternatively, be implemented in hardware and/or firmware.

The illustrative computing device 600 shown in FIG. 19 is not intended to suggest any limitation as to the scope of use or functionality of embodiments of the present disclosure. Neither should the illustrative computing device 600 be interpreted as having any dependency or requirement related to any single component or combination of components illustrated therein. Additionally, various components depicted in FIG. 19 may be, in embodiments, integrated with various ones of the other components depicted therein (and/or components not illustrated), all of which are considered to be within the ambit of the present disclosure.

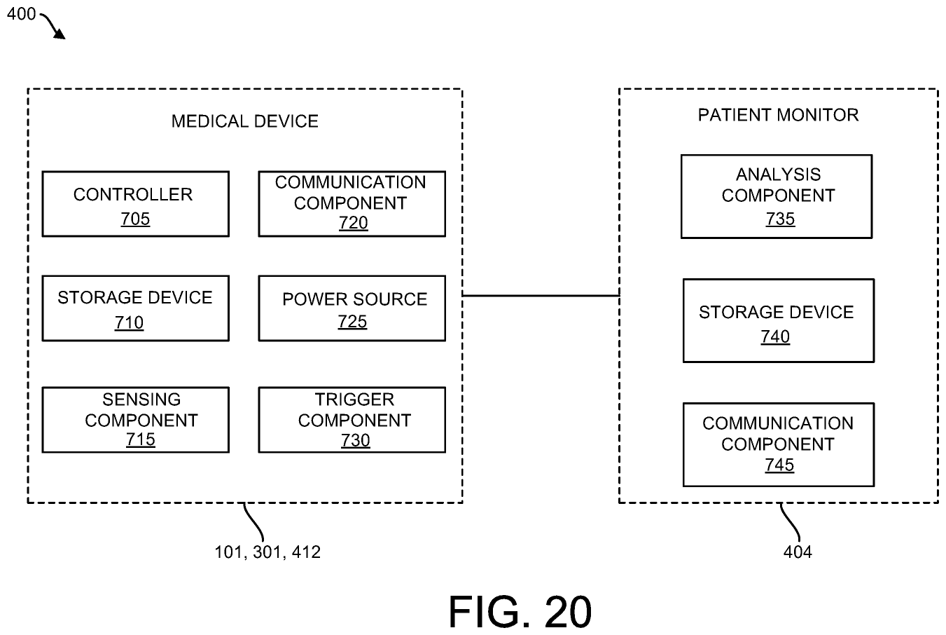

Referring to FIG. 20, there is shown a block diagram depicting a system 400 having an illustrative monitoring system 404 and medical device, which could be the catheter 101′, ultrasound device 412, the needle catheter 301′, and/or a combination of any of the foregoing in accordance with embodiments of the present disclosure. The medical device (e.g., 101, 301, 412) includes a controller 705, a storage device 710, a sensing component 715 (e.g., transducer), a communication component 720, a power source 725, and a trigger component 730. The controller 705 may include, for example, a processing unit, a pulse generator, and/or the like. The controller 705 may be any arrangement of electronic circuits, electronic components, processors, program components and/or the like configured to store and/or execute programming instructions, to direct the operation of the other functional components of the medical device (e.g., 101′, 412, 301′) to perform imaging, classification algorithms to determine the type of targeted tissue, algorithms to determine the location of the targeted tissue (including distance from other tissue or physiological structures), algorithms to determine the size of the targeted tissue size, and algorithms to determine the density of the targeted tissue, to store physiologic data obtained by the sensing component 715, algorithms to move and control the movement of the medical device, and/or the like, and may be implemented, for example, in the form of any combination of hardware, software, and/or firmware.

In embodiments, the controller 705 may be a programmable micro-controller or microprocessor, and may include one or more programmable logic devices (PLDs) or application specific integrated circuits (ASICs). In some implementations, the controller 705 may include memory as well. Although embodiments of the present system 400 are described in conjunction with a medical device 400 having a microprocessor-based architecture, it will be understood that the medical device (or other device) may be implemented in any logic-based integrated circuit architecture, if desired. The controller 705 may include digital-to-analog (D/A) converters, analog-to-digital (A/D) converters, timers, counters, filters, switches, and/or the like. The controller 705 executes instructions and performs desired tasks as specified by the instructions.

The controller 705 may also be configured to store information in the storage device 710 and/or access information from the storage device 710. The storage device 710 may be, be similar to, include, or be included within, the storage device 630 depicted in FIG. 19. That is, for example, the storage device 710 may include volatile and/or non-volatile memory, and may store instructions that, when executed by the controller 705 cause methods and processes to be performed by the medical device. In embodiments, the controller 705 may process instructions and/or data stored in the storage device 710 to control delivery of an electrical stimulation therapy by the medical device, to control sensing operations performed by the medical device, to control communications performed by the medical device, and/or the like.

The medical device may sense imaging signals using a sensing component 715 (or a plurality of sensing components) that may include, for example, one or more transducers 107′, 416 and/or one or more sensors (not shown), or a combination of these. The storage device 710 may be used to store sensed information according to some implementations. Information from sensing circuits included in the sensing component 715 may be used to adjust therapy, sensing, and/or communications parameters.

The communication component 720 may include, for example, circuits, program components, and one or more transmitters and/or receivers for communicating wirelessly with one or more other devices such as, for example, the monitoring system 404. According to various embodiments, the communication component 720 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 720 may include any combination of hardware, software, and/or firmware configured to facilitate establishing, maintaining, and using any number of communication links. In embodiments, the communication component 720 of the medical device facilitates wireless communication with the monitoring system 404. In embodiments, the communication component 720 may also facilitate communications with other medical devices such as, for example, to facilitate coordinated operations between the medical devices.

The power source 725 provides electrical power to the other operative components (e.g., the controller 705, the sensing component 715, the storage device 710, and the communication component 720), and may be any type of power source suitable for providing the desired performance and/or longevity requirements of the medical device. In various embodiments, the power source 725 may include one or more batteries, which may be rechargeable (e.g., using an external energy source). The power source 725 may include one or more capacitors, energy conversion mechanisms, and/or the like. Power sources for medical devices such as the medical device are well known, and are therefore not discussed in greater detail herein.

Continuing to refer to FIG. 20, the medical device may include a trigger component 730. In embodiments, the trigger component 730 may be implemented in any combination of hardware, software, and/or firmware, and may be implemented, at least in part, by the controller 705 of the medical device. The trigger component 730 is configured to detect a trigger event. According to embodiments, the trigger component 730 may be configured to implement any number of different adjudication algorithms to detect a trigger event. The trigger component 730 may detect a trigger event based on information received from any number of other components, devices, and/or the like. For example, the trigger component 730 may obtain imaging signals from the sensing component 730 and may use that physiological parameter signals to detect a trigger event. Trigger events may be user defined, system defined, statically defined, dynamically defined, and/or the like. The trigger component 730 may reference trigger criteria stored in memory (e.g., the storage device 710) to determine whether a trigger event has occurred. The trigger criteria may be established by a clinician, a patient, an algorithm, and/or the like.

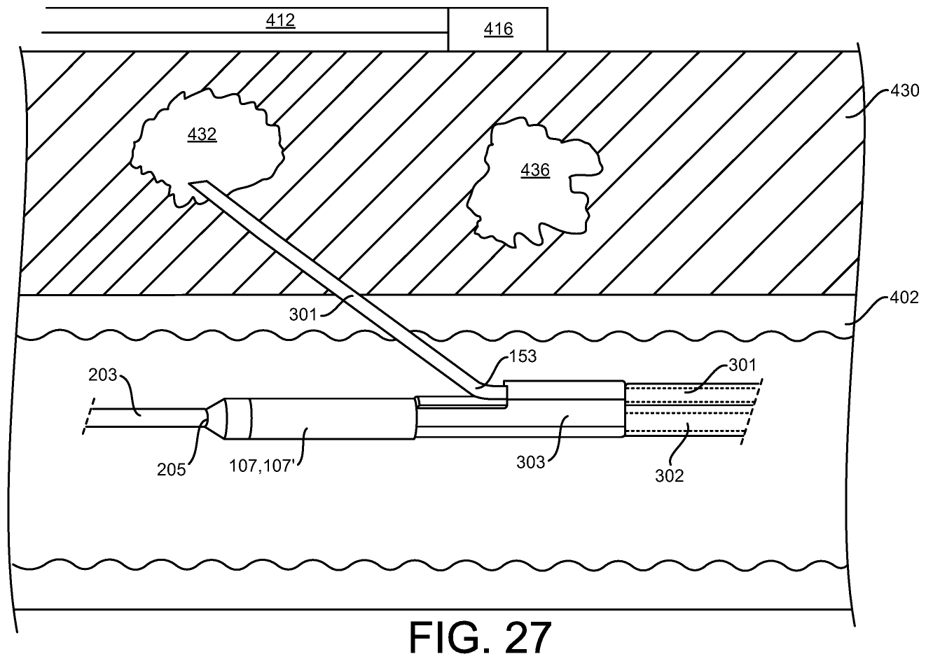

For example, the catheter 101′, the ultrasound device 412, and/or the needle catheter 301′ may be communicatively coupled to a trigger component 730. The trigger component 730 coupled to the catheter 101′ and/or the ultrasound device 412 may initiate the respective transducers 107′, 416 for those medical devices. The needle catheter 301′ may include a trigger component 730 illustrated as a trigger 414 (as shown in FIG. 18). Although the trigger 414 for the needle is depicted on or coupled to the needle catheter 301′ in this figure, the trigger 414 may be on the catheter 101′ if the catheter 101′ includes or is coupled to the needle 301′. The trigger 414 for the needle may be initiated by the clinician or the trigger 414 for the needle may be implemented when the trigger component 730 references a first set of trigger criteria for determining whether a first trigger event has occurred, a second set of trigger criteria for determining whether a second trigger event has occurred, and/or the like. The first trigger event may be, for example, when the needle catheter 301′ and/or the catheter 101′ is detected as being within a certain location in the vasculature and/or within a certain distance from the target tissue. Upon initiating the first trigger event, the needle extends through the vasculature and into the patient's soft tissue. The second trigger event may be, for example, when the needle is detected as being within the patient's targeted soft tissue, as illustrated in FIG. 27. Upon initiating the second trigger event, the needle begins to inject and deliver a therapeutic agent to the targeted soft tissue. The therapeutic agent may be included to the needle catheter 301′ or attached to the needle catheter 301′ via an adapter 418 (e.g., luer adapter), as shown in FIG. 18. A third trigger event may include the needle injecting a certain amount or dose of one or more therapeutic agents to the targeted soft tissue. Upon initiating the third trigger event, the needle discontinues injecting and delivering the therapeutic agent to the targeted soft tissue, and the needle is retracted from the targeted tissue, the patient's soft tissue, through the vasculature and back into the needle catheter 301′. A fourth trigger event may include completion of the needle back into the needle catheter 301′. Upon initiating the fourth trigger event, the needle catheter 301′ and/or the catheter 101′ is retracted from the vasculature.

Referring again to FIG. 20, the monitoring system 404 includes an analysis component 735, a storage device 740, and a communication component 745. In embodiments, the analysis component 735 may be implemented in any combination of hardware, software, and/or firmware, and may be implemented, at least in part, by a controller (not shown) that may be identical to, or similar to, the controller 705 of the medical device. Additionally, the storage device 740 and communication component 745 may be identical to, or similar to, the storage device 710 and the communication component 720, respectively, of the medical device. The monitoring system 404 may include any number of other components or combination of components including, for example, a sensing component, a therapy component, and/or the like. The analysis component 735 may perform or apply a more accurate (and therefore likely more computationally expensive) analysis than the trigger component 730 upon receiving information communicated to the monitoring system 404 from the medical device.

Referring to FIG. 18, FIG. 21 and FIG. 22 and FIG. 23, the transducer 107′ is attached to the end or distal portion of the catheter 101′ and maneuvered through a vascular structure 420 of a patient 424 to a point of interest. The transducer 107′ is then pulsed (see e.g., 428) to acquire echoes or backscattered data 422 reflected from the tissue of the vascular structure 420, as shown and discussed in U.S. Pat. No. 8,449,465 which is hereby incorporated by reference. Because different types and densities of tissue absorb and reflect ultrasound data differently, the reflected data (i.e., backscattered data) 422 can be used to image the vascular object. In other words, the backscattered data 422 can be used (e.g., by the monitoring system 404) to create an image of the vascular tissue (e.g., an IVUS image, a tissue-characterization image, etc.). For example, a first portion of backscattered data 422a might represent an inner portion of vascular tissue, a second portion of backscattered data 422b might represent a middle portion of vascular tissue, and a third portion of backscattered data 422c might represent an outer portion of the vascular tissue. In order to distinguish the different layers of vascular tissue, including occlusions and calcifications therein, the transducer 107′ may be driven in the frequency range of 500 kilohertz (KHz) to 25 megahertz (MHz).

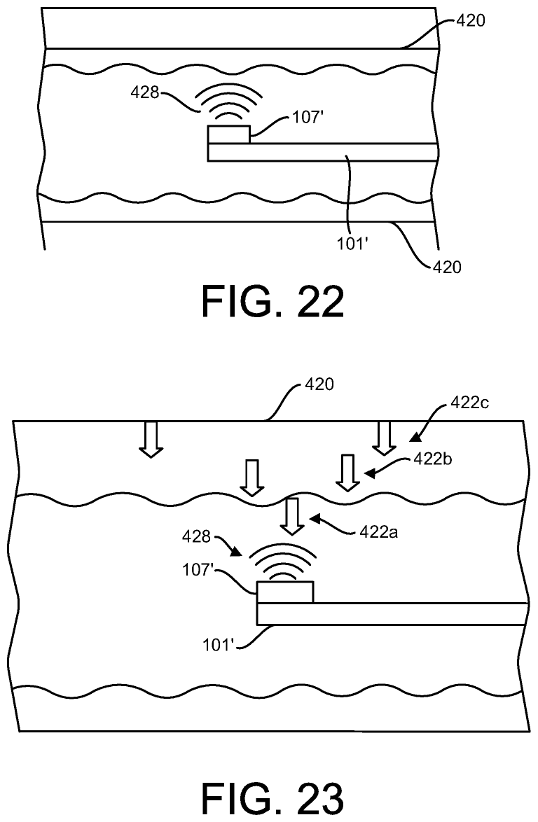

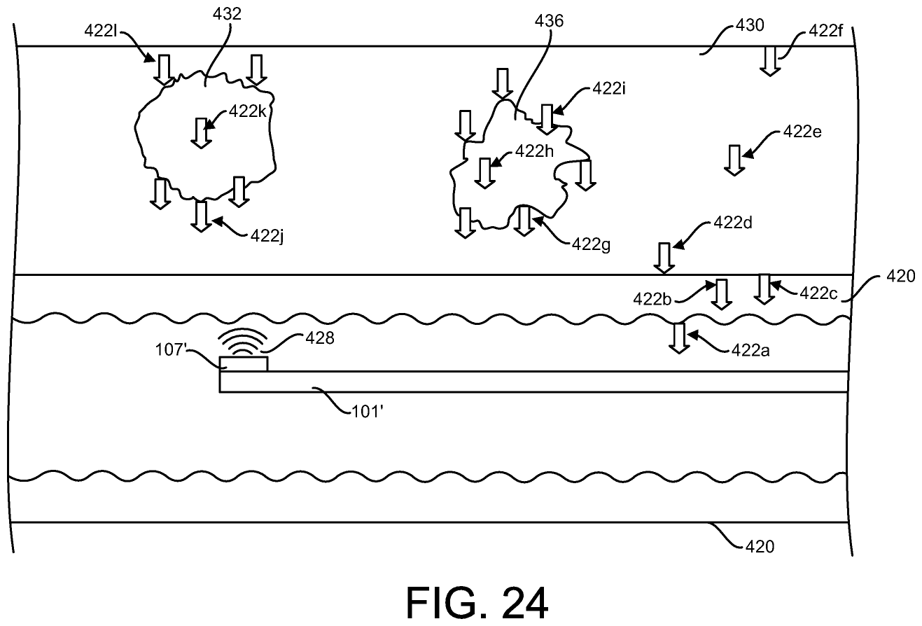

Referring to FIG. 24, there is shown an intravascular-ultrasound (IVUS) catheter 101′ having a transducer 107′ inside a patient's vasculature 420 and receiving ultrasound data as backscattered data. In addition to or in lieu of the transducer 107′ (in conjunction with the monitoring system 404) being configured to receive backscattered date to distinguish a first portion of backscattered data 422a that may represents an inner portion of vascular tissue, a second portion of backscattered data 422b that may represent a middle portion of vascular tissue, and a third portion of backscattered data 422c that may represent an outer portion of the vascular tissue, similar to that described with respect to FIG. 22, the transducer 107′ (in conjunction with the monitoring system 404) may also be configured to receive backscattered date to distinguish soft tissue 430 within a patient from the patient vasculature, wherein the soft tissue 430 is exterior of the vasculature 420 and exterior of the vascular tissue. The acoustic impedance of different types of tissue different. As such, the change between tissue types causes at least a partial reflection of the pulsed signal to reflect. That is, the discontinuity within the tissue or medium causes the signal (e.g., sound beam) emitted by the transducer to reflect. The reflected beam or data will be collected by the transducer which is called echo. From the echo, the pulse time can be calculated by multiplying the speed of sound with echo flight time, such as half of the double loop flight time. The thickness of the tissue can also be determined by calculating the difference between flight times for different echos. For example, echo 422c will take time (t₁) to travel from the inner portion of the soft tissue 430 (outer portion of the vasculature 420) to the transducer 107′, and echo 422a will take time (t₂) to travel from the inner surface of the soft tissue 430 to the transducer 107′. The difference between t₂ and t₁ is correlated to the thickness of the vasculature.

The transducer 107′ (in conjunction with the monitoring system 404) may also be configured to receive backscattered date to distinguish between different types of a soft tissue (e.g., tendons, ligaments, fascia, skin, fibrous tissues, fat, membranes muscles, nerves, etc.) from one another. The transducer 107′ (in conjunction with the monitoring system 404) may also be configured to receive backscattered date to distinguish between soft tissue from other structures, such as bone and nerves, within the patient's body. In order to distinguish the different layers of soft tissue, including other structures within the patient's body or soft tissue, the transducer 107′ may be driven in the frequency range of 500 kilohertz (KHz) to 30 megahertz (MHz), such as between 500 KHz and 25 MHz, between 500 KHz and 20 MHz, between 500 KHz and 15 MHz, between 500 KHz and 10 MHz, between 500 KHz and 9 MHz, between 500 KHz and 8 MHz, between 500 KHz and 7 MHz, between 500 KHz and 6 MHz, between 1 MHz and 5 MHz, between 1 MHz and 4 MHz, between 1 MHz and 4 MHz, between 1 MHz and 3 MHz, between 1 MHz and 2 MHz, and any value within such ranges.

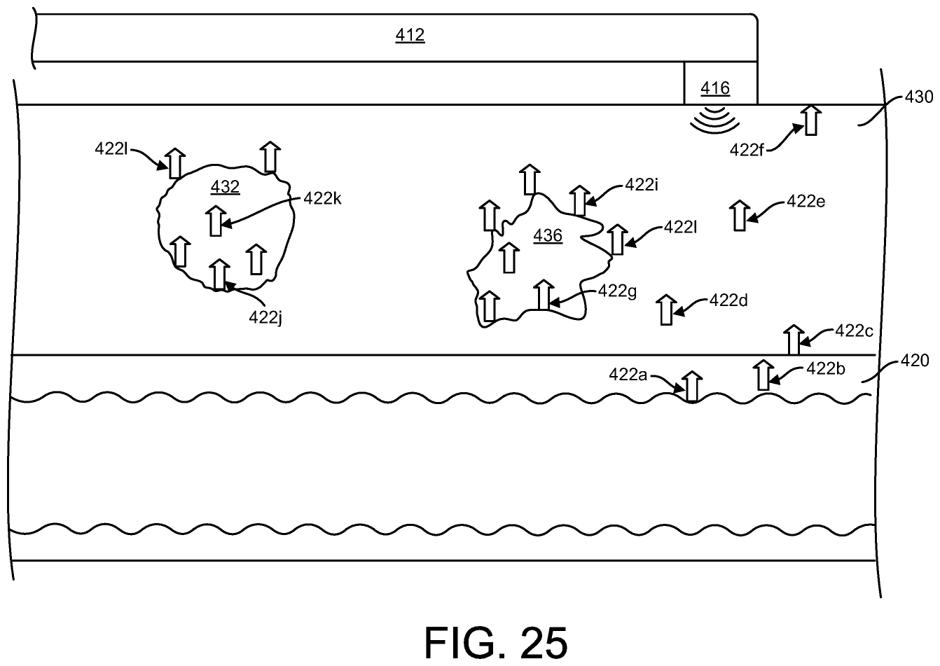

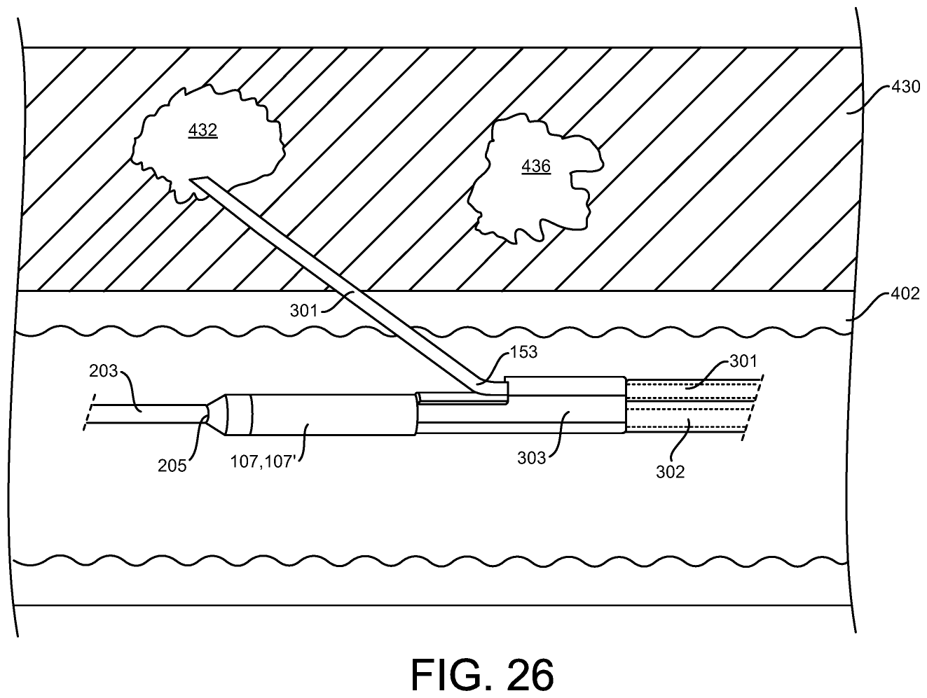

Referring to FIG. 24 and FIG. 25, the ultrasound transducers 107′, 416 may be inserted intravascularly or applied externally to the patient, respectively. Moreover, the ultrasound transducers 107′, 416 may both be inserted intravascularly and applied externally to the patient, thereby potentially increasing the accuracy during insertion of the needle. The ultrasound transducers 107′, 416 may receive a fourth portion of backscattered data 422d that may represents an inner portion of soft tissue, a fifth portion of backscattered data 422e that may represent a middle portion of soft tissue, and a sixth portion of backscattered data 422f that may represent an outer portion of soft tissue. For example, item 430 may represent the myocardium, and the fourth portion of backscattered data 422d may reflect off of the endocardium (or a layer between the endocardium and the vasculature), the fifth portion of backscattered data 422e may reflect off of a particular layer in the myocardium, and the sixth portion of backscattered data 422f off of the serous pericardium (or a layer between the myocardium and the pericardium). The flight time for each of the echos 422f and 422d can be calculated, and the difference between the flight times allows the thickness of the myocardium to be determined because the thickness of the myocardium is dependent upon and correlates to the difference in the echos 422f and 422d flight times. Based on the wall thickness of the myocardium, the amount of therapeutic agent applied to the myocardium can be determined and/or adjusted accordingly, thereby to achieving intelligent drug delivery to the myocardium based on the myocardium thickness. The therapeutic agents may be active pharmaceuticals, cytokines isolated using native extraction or recombinant technologies (growth factors, cell signaling molecules, etc.), immune cells, autologous or allogenic stem cell mixtures, bulking agents, adhesives, or denaturing chemicals.

The ultrasound transducers 107′, 416 may also receive additional portions of backscattered data 422g, 422h, 422i, that may represents various portions of soft tissue or physiologic structures 436, which should be avoided. The ultrasound transducers 107′, 416 may receive further portions of backscattered data 422j, 422k, 422l, that may represents various portions of targeted soft tissue 432, which is desirable for treatment using a therapeutic agent. Again, the backscattered data is reflected when there is a change in impedance. The impedance difference could be characterized with or without an imaging system. The sound wave generated by the transducer will be reflected back from the boundary between tissues (or between tissue and a physiologic structure) with different acoustic impedance. This signal could be received by transducer again, and showing an extra peak from pulse echo time response. The echo testing could be combined with imaging testing, but it can also be independent. Any screen showing the received pulse response could show the reflected signal from boundaries between tissues with different acoustic impedance, such as an oscilloscope.

The ability for the transducer 107′ and the monitor 404 to distinguish between different types of soft tissue and from other physiological structures provides the clinician with the ability to effectively guide a needle to a targeted tissue and deliver a therapeutic agent while increasing the ability and accuracy of inserting and delivering the needle to the targeted tissue while avoiding potential contact between the needle and non-targeted tissue or physiologic structures. Additionally, use of ultrasound devices, such as the catheter 101′ and ultrasound device 412 provides the clinician with information, such as the type, location, size and density, of targeted tissue. This information potentially allows the clinician to more successfully treat the patient by delivering the clinically effective amount and type of therapeutic agent, thereby increasing the likelihood of improved patient outcomes.

Referring to FIG. 26, there is shown a dual lumen imaging apparatus (similar to that described above with respect to FIGS. 11A-11D), with a first exit port having a needle 301 extending therefrom and through the vasculature 420 and delivering a therapeutic agent into a target 432 within the soft tissue 430 of a patient exterior of the vasculature 420 and exterior of the vascular tissue, while avoiding non-targeted 436 during needle insertion. Although FIGS. 11A-11D illustrate a dual lumen imaging apparatus, the apparatus my omit the imaging component or transducer 107, while retaining the other structure of the apparatus such that a dual lumen apparatus remains—one lumen 302 for the guidewire 203 and one lumen for the needle 301 to deliver the therapeutic agent. Referring to FIG. 27, there is shown an external imaging apparatus 412 and the dual lumen imaging apparatuses with a first exit port having a needle 301 extending therefrom and through the vasculature 420 and delivering a therapeutic agent into a target 432 within the soft tissue 430 of a patient exterior of the vasculature 420 and exterior of the vascular tissue, while avoiding non-targeted tissue or physiologic structures 436 during needle insertion.

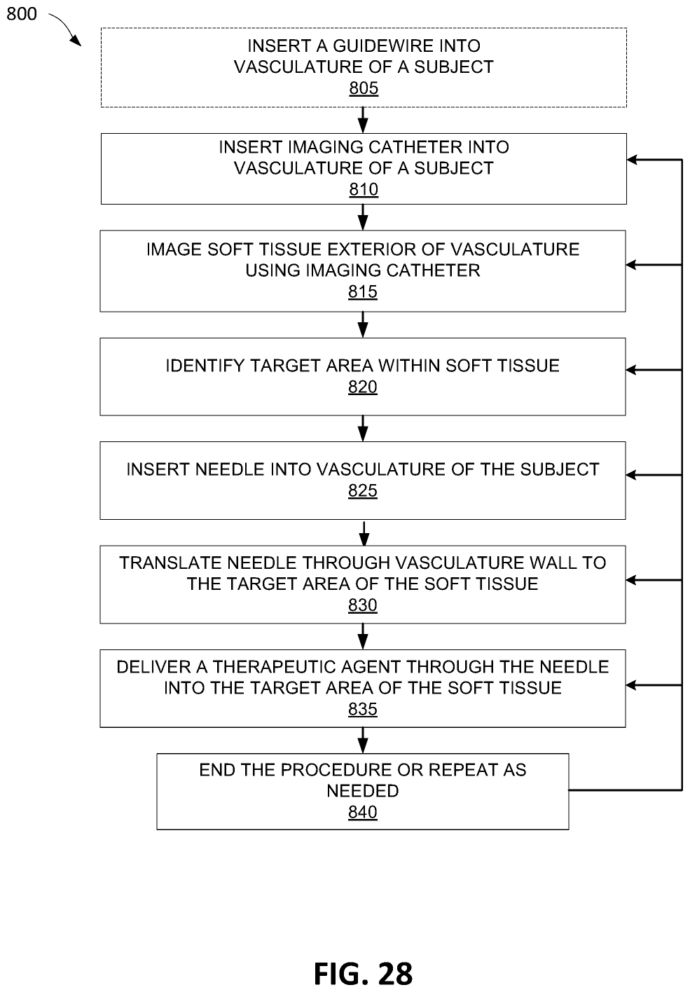

Referring to FIG. 28, there is shown a block diagram or flow chart of operating and/or using the device(s) discussed herein, such as the device(s) illustrated in FIG. 26. The dual lumen imaging device, such as imaging catheter 101′, may include both a guidewire lumen in addition to a lumen for delivering a therapeutic agent. If so, it may be desirable to initially insert a guidewire into the vasculature of a subject, as depicted in step 805 of FIG. 28. Step 810 includes inserting imaging catheter 101′ into vasculature of a subject by inserting the guidewire 203 into guidewire lumen 302 and sliding the imaging catheter 101′ over the guidewire 203. Step 815 includes using the imaging catheter 101′ to image the soft tissue located externally of the vasculature and externally of the vascular tissue. Step 820 includes identifying a target area within soft tissue. This step may include storing the imaging data collected from the imaging apparatus 107′ and using the stored imaging data and one or more algorithms to determine the type of targeted tissue, the location of the targeted tissue (including distance from other tissue or physiological structures), the size of the targeted tissue size, and the density of the targeted tissue. The imaging data may also be used to store other physiologic data, such as other tissue types and/or physiological structures to avoid during needle insertion. Additional algorithms may be used to move and control the movement of the imaging catheter 101′.

Continuing to refer to FIG. 28, once the distal portion of imaging catheter 101′ is located in the desirable position within the vasculature adjacent the target tissue, the needle 301′ is inserted into the vasculature as illustrated in step 825. For example, the needle 301′ may be inserted into the vasculature through the imaging catheter 101′, and the needle 301′ may extend from the imaging catheter 101′. That is, the needle 301′ may translate axially and/or radially with respect to the imaging catheter 101′, including relative to the distal portion of the imaging catheter 101′, in a linear and/or non-linear fashion, such that the needle 301′ extends from the distal portion of the imaging catheter 101′ and toward the vasculature. Referring to step 830, the needle 301′ translates through the vasculature wall into the soft tissue disposed exteriorly of the vasculature and to the target area of the soft tissue. Referring to step 835, once the open port of the needle 301′ is located within the targeted soft tissue, the therapeutic agent is injected through the needle 301′ and delivered to the targeted soft tissue. After the desirable or predetermined amount of therapeutic agent is delivered to the targeted soft tissue, the needle 301′ can be retracted from the soft tissue and vasculature into the imaging catheter 101′, and the imaging catheter 101′ and/or needle catheter 301′ may be removed from the vasculature or any of the preceding steps may be repeated, as shown in step 840.

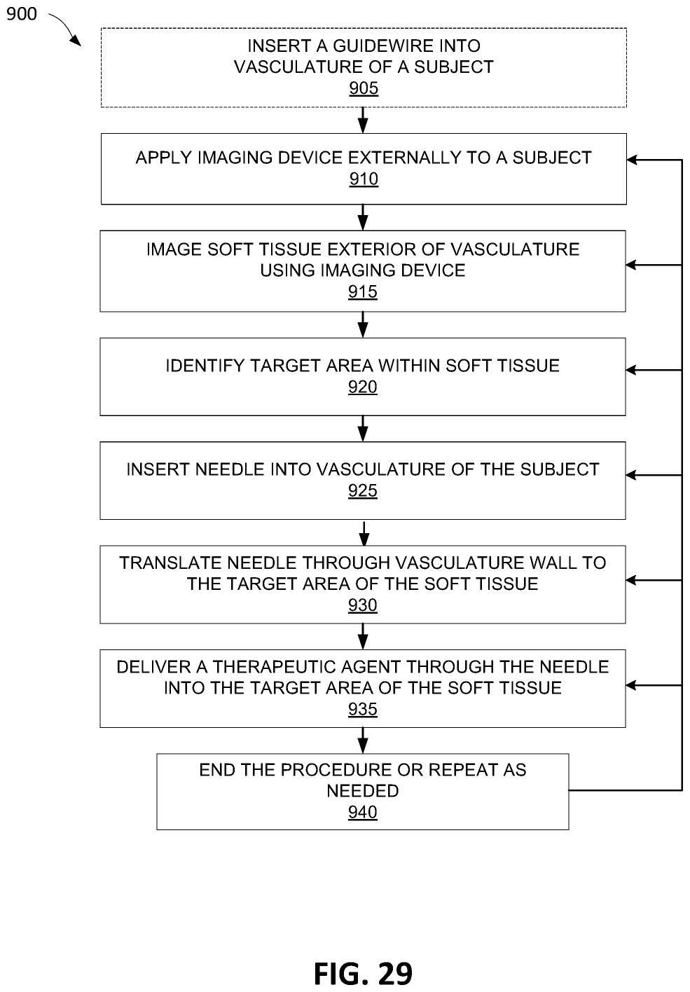

Referring to FIG. 29, there is shown a block diagram or flow chart of operating and/or using the device(s) discussed herein, such as the device(s) illustrated in FIG. 27. The method depicted in FIG. 29 is similar to the method depicted in FIG. 28 and described above, but the method in FIG. 29 uses an externally imaging device 412, such as that shown in FIG. 27 in lieu of the imaging catheter 101′ shown in FIG. 26 to image the soft tissue and identify the target area.

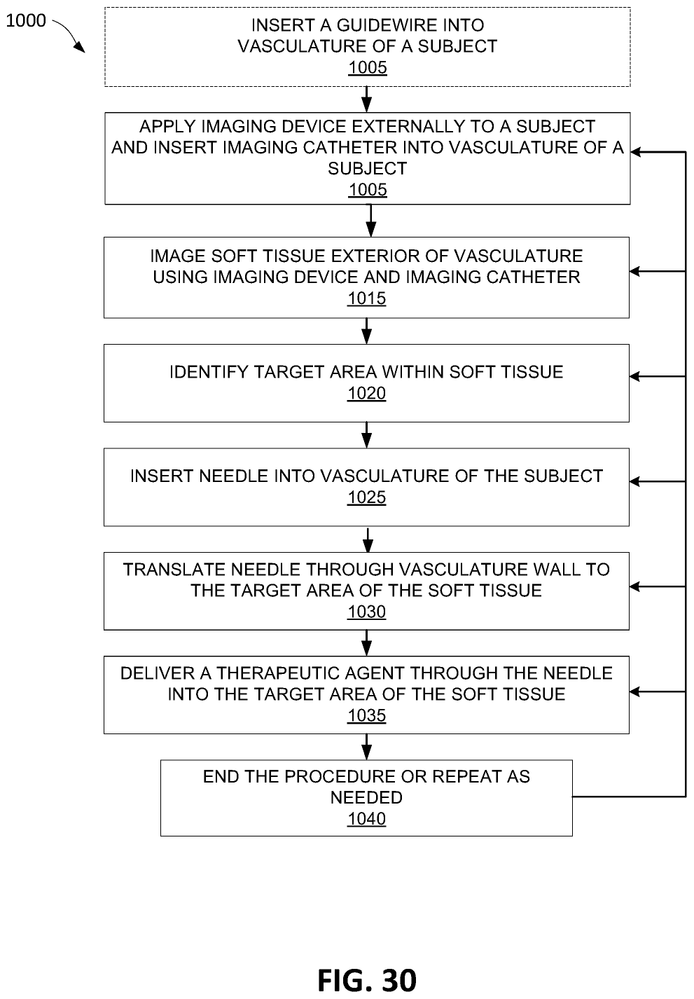

Referring to FIG. 30, there is shown a block diagram or flow chart of operating and/or using the device(s) discussed herein, such as the device(s) illustrated in FIG. 26 and FIG. 27. The method depicted in FIG. 30 is similar to the methods depicted in FIG. 28 and FIG. 29, but the method in FIG. 30 uses both imaging catheter 101′ and an external imaging device 412 to image the soft tissue and identify the target area.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An intravascular treatment system comprising:
a catheter comprising:
a distal portion and a proximal portion;
an imaging apparatus disposed at the distal portion of the catheter and configured to image a location within a subject's soft tissue disposed externally of vasculature, the imaging apparatus producing an image signal; and
a first lumen comprising a first exit port disposed at the distal portion of the catheter, wherein the first lumen is configures to receive a guidewire; and
a needle slidably disposed within the catheter, wherein the needle comprises a second lumen and a second exit port; and
a controller for receiving the image signal, the controller comprising a non-transitory computer-readable medium containing instructions that, when executed, cause one or more processors to:
image the subject's soft tissue disposed externally of vasculature using the image signal;
identify a target area within the subject's soft tissue;
translate the needle relative to the catheter and inserting the needle through the vasculature to the target area; and
deliver a therapeutic agent to the target area through the needle.

2. The system of claim 1, wherein the non-transitory computer-readable medium's instructions for identifying the target area comprise instructions that, when executed, cause one or more processors to determine a type of tissue within the target area.

3. The system of claim 1, wherein the non-transitory computer-readable medium's instructions for identifying the target area comprise instructions that, when executed, cause one or more processors to determine a location or position of the target area within the subject's soft tissue.

4. The system of claim 3, wherein the location or position of the target area comprises distance.

5. The system of claim 4, wherein the distance is relative to another portion of the subject's soft tissue.

6. The system of claim 1, wherein the non-transitory computer-readable medium's instructions for identifying the target area comprise instructions that, when executed, cause one or more processors to identify a size of the target area.

7. The system of claim 1, wherein the non-transitory computer-readable medium's instructions for identifying the target area comprise instructions that, when executed, cause one or more processors to identify a density of the target area.

8. The system of claim 1, wherein the instructions for delivering the therapeutic agent to the target area through the needle comprise instructions that, when executed, cause one or more processors to deliver an amount of therapeutic agent based upon at least one of a size of the target area and a density of the target area.

9. The system of claim 1, wherein the image signal is produced from a transducer producing energy between 500 kilohertz (KHz) to 30 megahertz (MHz).

10. The system of claim 1, wherein the needle is substantially parallel to at least a portion of the first lumen of the catheter.

11. A method of treating a patient, wherein the patient comprises tissue disposed below skin and exterior of vasculature, the method comprising:
providing a catheter, wherein the catheter comprises:
a distal portion and a proximal portion;
an imaging apparatus disposed at the distal portion of the catheter and configured to image a location within a patient's soft tissue disposed externally of vasculature, the imaging apparatus producing an image signal; and
a first lumen comprising a first exit port disposed at the distal portion of the catheter, wherein the first lumen is configured to receive a guidewire; and
providing a needle slidably within the catheter and substantially parallel to at least a portion of the first lumen of the catheter, wherein the needle comprises a second lumen and a second exit port; and
imaging the patient's soft tissue disposed externally of vasculature using the image signal;
identifying a target area within the patient's soft tissue;
translating the needle relative to the catheter and inserting the needle through the vasculature to the target area; and
delivering a therapeutic agent to the target area through the needle.

12. The method of claim 11, wherein identifying the target area comprises determining a type of tissue within the target area.

13. The method of claim 11, wherein identifying the target area comprises determining a location or position of the target area within the patient's soft tissue.

14. The method of claim 13, wherein the location or position of the target area comprises distance.

15. The method of claim 14, wherein the distance is relative to another portion of the patient's soft tissue.

16. The method of claim 11, wherein identifying the target area comprises identifying a size of the target area.

17. The method of claim 11, wherein identifying the target area comprises identifying a density of the target area.

18. The method of claim 11, wherein delivering the therapeutic agent to the target area through the needle comprises delivering an amount of therapeutic agent based upon at least one of a size of the target area and a density of the target area.

19. The method of claim 11, wherein the imaging apparatus is a transducer producing energy between 500 kilohertz (KHz) to 30 megahertz (MHz).

20. The method of claim 11, wherein the needle is substantially parallel to at least a portion of the first lumen of the catheter.
